(19) United States
(12) Patent Application Publication
(10) Pub. No.: US 2018/0250504 A1
(43) Pub. Date: Sep. 6, 2018

(54) SYSTEM FOR MEDICAL INTRAVASCULAR LINE CONTAMINATION PROTECTION

(71) Applicant: Joseph P. Schultz, Atlanta, GA (US)

(72) Inventor: Joseph P. Schultz, Atlanta, GA (US)

(21) Appl. No.: 15/973,177

(22) Filed: May 7, 2018

Related U.S. Application Data
(63) Continuation-in-part of application No. 15/971,091, filed on May 4, 2018.
Application No. 15/971,091 is a continuation of application No. 14/208,487, filed on Mar. 13, 2014, now Pat. No. 9,974,941.
(60) Provisional application No. 62/502,284, filed on May 5, 2017.
(60) Provisional application No. 61/779,122, filed on Mar. 13, 2013.

(51) Int. Cl. A61M 39/16; A61M 39/10

(52) U.S. Cl. CPC A61M 39/165; A61M 39/1011; A61M 2039/1066; A61M 2039/1033

(57) ABSTRACT

The present invention provides better protection to IC connection joints during assembly, access, connection, and during maintenance. The present disclosure relates to a protection barrier for an IV access system joint of an IV access system, so that the IV access system and patient are better protected from environmental contamination and microbial ingress between access events or while maintained in a connected configuration.

### CROSS-REFERENCE TO RELATED APPLICATION

This application claims priority to, and the benefit of, U.S. Provisional Application No. 62/502,284, filed May 5, 2017, the entirety of which is hereby incorporated by reference; and this application is a Continuation-in-Part of, and claims priority to, and the benefit of, U.S. patent application Ser. No. 15/971,091, filed May 4, 2018, which is a continuation of U.S. patent application Ser. No. 14/208,487, filed Mar. 13, 2014, which claims the priority benefit of U.S. Provisional Patent Application Ser. No. 61/779,122, filed Mar. 13, 2013, each of which are hereby incorporated herein by reference.

### TECHNICAL FIELD

The present disclosure relates generally to reducing touch contamination during intravascular assembly and access, and more specifically to incorporating a removable open ended shield to protect intravascular connection joints.

### BACKGROUND

There is a big public health and patient safety need to reduce bloodstream infections in patients, including those associated with central lines and peripheral intravenous (IV) catheters. Additionally, gaps that can occur at intravascular (IV) connector joints and hub assemblies over time provide access for debris, contaminants and migrating microorganisms. Little attention has been made to protecting IV lines and needleless access valves to protect them from contamination while assembled for IV administration or disassembled with a free end during storage or maintenance. During prolonged maintenance, contaminates can attach to the system, and microbial growth can occur over time and biofilm can develop.

### SUMMARY

The present invention provides better protection to IC connection joints during assembly, access, connection, and during maintenance. The present disclosure relates to a protection barrier for an IV access system joint of an IV access system, so that the IV access system and patient are better protected from environmental contamination and microbial ingress between access events or while maintained in a connected configuration.

An example embodiment apparatus to surround a male luer connector pre-assembled to a compatible threaded female connector, (such as a connector with a female lock connector or a needleless access valve with female luer threads), in fluid communication with an intravascular tubing entering a patient's body, to provide prolonged contamination protection to the outer surfaces of the assembled parts to prevent ingress of contamination into the inner surfaces of the intravascular tubing, the apparatus including a pod oriented around a longitudinal axis, the pod including a chamber with an inner chamber surface and an outer wall with an outer wall surface and an inner chamber length greater than the shortest combined length of a standard male luer lock and a standard female luer lock. The example embodiment chamber has a proximal end and a distal end and an opening thought the inner chamber surface to the outer wall surface from the proximal end toward the distal end of the chamber to allow insertion of a male luer connector pre-assembled to the compatible threaded female connector into the chamber from a position parallel to the longitudinal axis. The example embodiment apparatus has least one exit port for intravascular tubing. The example embodiment pod forms a sealed chamber around the entire length of the male luer connector and compatible female connector. The example embodiment exit port seals around the IV tubing. The example embodiment chamber includes absorbent material. The example embodiment chamber includes permeable material. The example embodiment chamber includes antibacterial solution or material with antibacterial properties. The example embodiment chamber is permeable to gases. The example embodiment chamber is permeable to liquids. The example embodiment chamber includes material soaked in an antibacterial solution. The example embodiment pod includes an end cap. The example embodiment pod includes an end cap with an exit port. The example embodiment pod includes an end cap without an exit port. The example embodiment pod includes a first end cap with a port and a second end cap with a port. The example embodiment pod includes a single monolithic part. The example embodiment pod includes a bi-valved construction. The example embodiment pod includes a tube. The example embodiment pod includes a pocket with a dead end. The example embodiment pod includes a test tube. The example embodiment apparatus includes an inner chamber surrounding an assembled male luer lock and a compatible female connector. The example embodiment apparatus includes an inner chamber surrounding an assembled male luer lock of first medical device and a compatible female connector. The example embodiment apparatus includes an inner chamber surrounding a female connector of a needleless access valve and an assembled male luer lock of the needless access valve, assembled to a compatible female connector attached to an intravascular lined. The example embodiment apparatus includes an inner chamber surrounding an intravenous line with a male luer lock preassembled to female connector of a needleless access valve and an assembled male luer lock of the needless access valve, assembled to a compatible female connector attached to an intravascular line.

An apparatus for protecting a patient by providing a protection barrier for an intravascular access system joint (IV joint) of an intravascular access system, so that the intravascular access system and patient are better protected from environmental contamination and microbial ingress between access events/while maintained in a connected configuration; the intravascular access system joint comprising an intravascular (IV) access line having a flexible IV access tube with a distal portion entering into a patient and a proximal portion bonded at the proximal end to a female luer hub; a first IV device with a first hub connector having a first female luer connector with a minimum standard luer connector length; the first female luer connector is connected to a first male luer connector of a second IV device hub connector of a second IV medical device. The apparatus can include a protective barrier that encircles the IV joint along the longitudinal axis of the first and second hubs to form a chamber enclosing the external joint space between the first medical device and second medical device. The apparatus can include a pod with a pod inner chamber. The pod is structured and arranged to be removable without breaking the assembly. The pod inner chamber encloses the IV joint and a needless access valve (with a male luer lock and female luer threads). The pod inner chamber encloses at least two IV connector assemblies, each with a male luer and female luer connection. The apparatus can also include a pod with a port for a first IV tube. The apparatus can also include a pod with two ends; and a first port for a first IV tube and a second port for a second IV tube located at opposite ends of the pod. The pod inner chamber encloses two IV tubes. The pod inner chamber encloses the IV joint between the IV line and a needless access valve with a male luer lock and female luer threads connected to a proximal IV line. The pod can also include a foam wall with an absorbent inner surface circumferentially surrounding the IV joint along the longitudinal axis. The pod can also include a hinge. The pod can be split open down the longitudinal axis.

An apparatus for protecting a patient by providing a protection barrier for an intravascular access system joint (IV joint) of an intravascular access system, so that the intravascular access system and patient are better protected from environmental contamination and microbial ingress between access events/while maintained in a connected configuration; the intravascular access system joint comprising an intravascular (IV) access line having a flexible IV access tube with a distal portion entering into a patient and a proximal portion bonded at the proximal end to a female luer hub; a first IV device with a first hub connector having a first female luer connector with a minimum standard luer connector length; the first female luer connector is connected to a first male luer connector of a second IV device hub connector of a second IV medical device. The apparatus comprising: a protective barrier that encircles the IV joint along the longitudinal axis of the first and second hubs to form a chamber sealing the external joint space between the first medical device and second medical device from external contamination. The apparatus can include a pod with a pod inner chamber. The pod can be structured and arranged to be removable tangentially from the longitudinal axis without breaking the IV joint assembly. The pod inner chamber can enclose the IV joint and a needless access valve, for example with male luer lock and female luer threads. The pod inner chamber can enclose at least two IV connector assemblies, each with a male luer and female luer connection. The pod inner chamber can encloses two IV tubes. The pod inner chamber can enclose the IV joint between the IV line and a needless access valve with a male luer lock and female luer threads connected to a proximal IV line. The pod can also include a port for a first IV tube. The pod can also include two opposite ends, and a first port for a first IV tube and a second port for a second IV tube located opposite each other at opposite ends of the pod. The pod inner chamber can also have antimicrobial properties. The pod can also include an absorbent inner surface impregnated with an antimicrobial solution and a smooth non-absorbent outer surface.

A system for protecting a patient by providing a protection barrier of connections in an intravascular access lines. The system comprising an IV joint, the IV joint comprising a line for intravascular (IV) access having a flexible IV access tube with a distal portion entering into a patient and a proximal portion bonded at the proximal end to a first female luer hub having a first female luer connector and connected to a distal male luer connector of a needleless access valve with a proximal female thread; a removable protective barrier forming a pod that begins distal to the IV joint and extends beyond the female threaded end of the needleless access valve, along the longitudinal axis of the first and second hubs. The pod comprises a pod inner chamber that encapsulates the external joint space between the first medical device and second medical device and the needless access valve. The first medical device is a CVL device. The pod can also include a resilient inner material surrounded and compressed by a rigid outer material. The pod can also include a side opening to split the pod open to facilitate its removal tangential to the longitudinal axis of the IV joint (without requiring disassembly of an assembled IV joint assembly); and, the pod inner chamber encloses the IV joint to seal it from external contamination and comprises a resilient inner surface with antimicrobial properties. The pod inner chamber can span IV joint and another connection surface (2 in 1, e.g. line or cap). The pod inner chamber can span two IV tubes. It can also include absorbent material with slit

A system for protecting a patient by providing a protection barrier of connections in an intravascular access lines. The system comprising an IV joint, the IV joint comprising a line for intravascular (IV) access (***CVL) having a flexible IV access tube with a distal portion entering into a patient and a proximal portion bonded at the proximal end to a first female luer hub having a first female luer connector and connected to a first male luer connector of a second IV device hub connector of a second IV medical device; a removable protective barrier that at least spans the IV joint along the longitudinal axis of the first and second hubs to form a chamber encircling and enclosing the external joint space between the first medical device and second medical device.

### BRIEF DESCRIPTION OF THE DRAWINGS

FIG. 1A is a schematic diagram of an example standard female luer lock connector and an intravenous line, such as a central line or peripheral IV line that goes to a patient.

FIG. 1B is a schematic diagram of an example IV valve such as a needleless access valve in connection with a male luer lock connector and an IV line.

FIGS. 1C and 1F are schematic diagrams of the example needleless access valve in FIG. 1B.

FIGS. 1D and 1E are schematic diagrams of the example male luer lock in FIG. 1B.

FIG. 1G is a schematic diagram of an example cap for a female luer lock.

FIG. 2A is a schematic diagram of an example standard female luer lock connector attached to a first intravenous line, such as a central line or peripheral IV line that goes to a patient and assembled with a needleless access valve and assembled with a male luer lock in connection with a second IV line for supplying fluids. The joint between the female luer lock connector and the needleless access valve; as well as the joint between the needleless access valve and the male luer lock are unprotected from contamination.

FIG. 2B is a schematic diagram of an example standard female luer lock connector attached to a first intravenous line, such as a central line or peripheral IV line assembled with a male luer lock in connection with a second IV line for supplying fluids. The joint between the female luer lock connector and the male luer lock is unprotected from contamination.

FIG. 2C is a schematic diagram of an example standard female luer lock connector attached to a first intravenous line, such as a central line or peripheral IV line that goes to a patient and assembled with a needleless access valve. The joint between the female luer lock connector and the needleless access valve is unprotected from contamination.

FIG. 2D is a schematic diagram of an example standard female luer lock connector attached to first intravenous line, such as a central line or peripheral IV line that goes to a patient and assembled with a cap. The joint between the female luer lock connector and the cap is unprotected from contamination.

FIG. 3A is a schematic diagram of an example plug with a groove in the sidewall and a tube, such as a test tube, separated from each other.

FIG. 3B is a schematic diagram of the example plug and tube shown in FIG. 3A, with the plug inserted into the tube.

FIGS. 3C, 3E, 3F and 3G are schematic diagrams of the plug and tube shown in FIGS. 3A and 3B with an IV line and IV connector inserted within. The IV line can be insert plug and then into the tube while connected to an IV device such as cap or needless access valve. By placing the tube over the device with the IV connector and over the IV line surrounded by the plug, the tube and plug now provide a protective sealed chamber which can now protect the assembled IV devices from touch contamination and other types of contamination.

FIG. 3D is a top view of the plug shown in FIG. 3A, illustrating the groove. A cross-sectional view can be seen of the plug with the groove that can have walls touching with a potential space or can have a gap

FIGS. 4A-4F are schematic diagrams of an example protective device with a tube and an inner chamber with an antimicrobial material such as antibacterial solution such as isopropyl alcohol or chlorhexidine gluconate (CHG) and an IV connector in FIGS. 4B-4F. There can be an evaporated or aerosolized gas within the tube as well.

FIGS. 5A-5F are schematic diagrams of an example tube, a plug and protective chamber that is formed by a compliant material such as a spongy material. The material can be absorbent or it can be permeable. It can be non-absorbent and it can be non-permeable. There can be a groove in the side or center that permits insertion of the assembled medical devices for protection into an inner chamber. The chamber can be sealed on at least one end, or both ends. The sealed chamber can be permeable or impermeable. FIGS. 5B-5F show the protective chamber protecting IV connectors.

FIGS. 6A-6D are schematic diagrams of an example assembled medical device with the IV joint between the needleless access valve and the female luer lock protected within the inner chamber of the tube, such as the tube described above. The inner chamber can have an antimicrobial solution such as isopropyl alcohol, CHG or other agents. The inner chamber can contain an antibacterial aerosol or gas. The solution, aerosol, gas or other material can leave outer surfaces of the assembled medical device coated to prevent infection or can leave sterile. Outer surfaces of the devices, such as those within a male luer lock would have superior protection to the prior art since these areas can not be easily reached with standard cleaning techniques during maintenance, connection or disconnection. The gas can permeate through a permeable surface of the inner chamber such as a permeable plug. The chamber can have a transparent wall to assess the contents and the condition of the contents inside. The inner chamber can contain an impregnated antibacterial material such as an alcohol swab that can evaporated and kill bacteria within. The alcohol can be soaked up by an absorbent material or can escape as a gas through a permeable material to reduce the risk of the chemical compounds have any negative stress effects on the medical device plastics over a prolonged period of time.

FIGS. 7A-7B show schematic diagrams of an example assembled medical device with the joint protected within the inner chamber within a tube such as the tube illustrated above. The inner chamber can have an antibacterial solution such as isopropyl alcohol, CHG or other agents. The inner chamber can contain an antibacterial aerosol or gas. The solution, aerosol, gas or other material can leave outer surfaces of the assembled medical device coated sterile. The solution or gas can be contained within the inner chamber which can be comprise impermeable walls that are resistant to the escape of the antibacterial solution. The inner chamber can contain an impregnated antibacterial material within the walls itself (FIG. 7A) or impregnated or soaked on separate material (FIG. 7B), e.g. an isopropyl alcohol swab, that can evaporated and kill bacteria within. The materials chosen can have protective properties that will reduce the risk of the chemical compounds having any negative stress effects on the medical device plastics over a prolonged period of time.

FIGS. 8A-8B are schematic diagrams of an example protective pod with an inner chamber can have two removable ends for use with a medical device assembly with a first and a second IV line at opposite ends.

FIGS. 9A-9B are schematic diagrams of an example protective pod with an inner chamber formed within a body that can be monolithic and can have two exit openings. The groove and the exit openings can be pre-formed to fit over the IV line or the inner chamber and exits can be formed as the inner surfaces contour over the outer surfaces of at least one of the medical devices. There can be clamp or other restraining elements that maintains the walls of the inner chamber in their optimal positions such as shown in the middle portion of the outer wall surface. An IV line preassembled to at least one medical device can be placed through the groove and into the inner chamber which can be formed by material of the inner wall which would then be surrounding the contours of the assembled medical devices. The preassembled medical device can have both a first and a second IV line that can be a different positions, such as at opposite ends forming a substantially linear line assembly with two IV lines exiting different ports formed or preformed at opposite ends. This embodiment, like others described here, would permit a protective chamber with protective walls and at times protective substances to be applied to assembled medical devices without the risks associated with disconnection of those medical devices.

FIGS. 10A-10B are schematic diagrams of an example protective pod with an inner chamber formed within a body that can be monolithic and can have two exit openings formed by a spongy, compliant, flexible, absorbent or permeable material. The groove or the exit openings can be preformed to fit over the IV lines; or the inner chamber and exits can be formed as the material contours over the outer surfaces of at least one of the medical devices. The pod can consist essentially of a monolithic body with or without an antibacterial material.

FIGS. 11A-11E are schematic diagrams of an embodiment with an example clamp or other restraining elements that maintains the wall of the inner chamber in their optimal positions after an assembled medical device with at least a first IV line is placed within. Two IV line are shown pre-assembled. The device and other embodiment demonstrate that the assembled connectors do not need to be separated or have a free end in order to apply the protection device. This reduces the contamination risk associated with assembling free connector ends and the problems associated with leakage of the contents of a working IV line including waste, leakage, resetting flow monitors and inaccurate dosing of administer medications that are problems in the prior art. FIGS. 11B-11E also show embodiments with an impregnated solution. The solution can be pre-applied by the manufacturer or applied at the time of application about the IV assembly. It can be reapplied during a maintenance phase or just prior to removal. The solution can retained in place by an absorbent material or an antibacterial material can be impregnated into the material to protect against microbial contamination. The solution can evaporated leaving the inner chamber sterile or cleaner and the walls of the chamber can go from a moist material to dry material during this process. The material can include either super hydrophobic or super hydrophilic properties that would provide antimicrobial protection in either a wet or dry state. One such material is a material with “DAC” that is marketed by BSN Medical Inc. which binds bacteria when wet and prevents the bacteria from moving.

FIGS. 12A-12B are schematic diagrams of example embodiments with an outer protective layer that can be impermeable to protect against ingress of contamination and to retain an antimicrobial substance within the chamber. A protective layer can provide a seal around the assembly to reduce any entrance or exit through the pod outer surface to the inner channel of the assembled IV line or lines. While linear connections are shown, other devices, such a stopcocks or y-connectors (not shown) with multiple ports and non-linear connections might also be protected in the same way by the present invention. Embodiments of the device could easily be assembled and removed to assembled IV connectors, including assembled IV lines with IV connectors; e.g. much easier than a tape wound around the connections as has been done in the prior art to protect against disconnections. Embodiments of the present invention can also provide additional friction to the surface of the connected devices within the inner chamber and this can help prevent disconnection and the hazards associated with these events.

FIGS. 13A-13C are schematic diagrams of example different embodiments of the present invention. The apparatus can contain a hollow shell with preformed exit ports. The shell can be comprised of at least two valves or components that assemble together. (FIG. 13A). The valves can be hinged to assist with retention and proper placement. The shell can have a softer inner material to compress against a variety of medical devices with different contours. (FIG. 13B). Embodiments can have a pre-formed recess for a more precise positioning, fitting and engagement with the outer contours of two or more assembled IV devices and can fit over multiple device with at least two IV lines that exit out opposite ends of the protective inner chamber. (FIG. 13C). The material can have properties described elsewhere in this description such as the antimicrobial or permeability properties. Multiple embodiments are envisioned with a variety of recesses and restraints that could fit specific combinations of models of medical connectors for ideal positioning, sealing and retention.

FIGS. 14A-14B are schematic diagrams of an example protective shell and inner wall with shrinking properties so that they can more tightly fit around the assembled IV device for better retention and sealing for improved protection of the devices. This might be achieved with a heat shrinkable material that is applied around the connected devices. Heat could be applied after application to shrink and seal the material and form a tighter more protective inner chamber. The shrink material can have antimicrobial properties that protect against contamination other than the physical barrier provided. The figure shows a cross sectional view of a wider pod (FIG. 14A) and a more narrow shrunken pod (FIG. 14B).

FIGS. 15A-15D are schematic diagrams of an example hinged bivalve shell with a soft material on the inside with at least one preformed recess for an assembled medical device. The assembled female luer lock, a needleless access valve and a male luer lock configuration with a supply IV line and a patient delivery IV line is placed within the pod though the open space along a longitudinal axis and when the bivalve shell is closed the assembled devices are protected by a sealed enclosure with an outer impermeable shell, an inner softer material impregnated with an antibacterial substance that is delivered to the outer surfaces of the medical device so that the inner material forming the chamber can provide surface antimicrobial protection and device retention. As shown in this embodiment and many others shown, the end openings can be smaller than the largest cross-sectional width of the connectors, smaller than the outer surface of male luer lock collar, smaller than the largest clearance of the inner threads of a standard ISO compliant male luer lock collar inner surface, smaller than the outer surface of a female luer lock threaded connector end, smaller than the distal tip of a male luer lock or smaller than the width of the IV tubing. In cases where an exit is smaller than a dimension listed, the opening can crimp the device or expand to accommodate it. For example, if the exit is smaller than the outer diameter of an IV tube, the material could clamp the outer surface of the IV tubing so it becomes smaller preferably without obstructing flow through the inner fluid channel and so the tubing is gripped well with a good outer seal over the tubing or the exit opening can expand to fit the size and contour of the outer wall of the IV tubing being stretched to form a good seal around the tubing.

FIGS. 16A-16D are schematic diagrams of an example connector with male and female ends. The connector has at least one circumferential shield at either a male or a female end that creates a circumferential seal over at the ends of at least two connectors at the position where they are engaged. The shield can be applied during connection of the connectors and can assist with targeting of the connectors and protect from touch contamination during assembly of the male and female connector devices. The shield can have a slit in a sidewall along the longitudinal axis to allow the shield to be applied or removed after assembly or to provide for application of the shield from the side, rather than over the tip of the connector, to prevent disconnecting and opening the line. Application or removal from the side could also reduce the risk of accidental touch contamination of the critical connector end or tip surface with a non-sterile surface during manipulation.

FIGS. 17-18 are schematic diagrams illustrating how a shield of the present invention can be delivered during assembly by one of the luer connectors, for example attached to the male connector end and delivered over the female luer connector end. There can be a pre-formed opening or the opening can be a potential space formed by the insertion of the connector on the receiving side. The end can be used for cleaning prior to insertion of the connector. There can be an open end of the shield with an inner clearance equal larger than the threads of a male luer lock collar or equal to or larger than the outer collar surface of a male luer lock collar or wide bodied needleless valve connector with a female connector with female luer lock threads that are compatible with a male luer lock. The shield can be removable from the male luer lock connector or the device with the male luer lock connector. The shield material can be absorbent to soak up blood or other material on assembly with a dripping IV hub or to soak up or deliver an antimicrobial substance to keep the shield and shielded connectors from becoming contaminated. If the shield were to get contaminated it can be constructed so that it can be removed from the side, e.g. if contaminated by blood after assembly of the connectors, and this could be accomplished without disconnection of the connectors. The shield with absorbent material can contain antibacterial properties and form a circumferential shield that can physically block contamination and can provide other anticontamination properties, e.g. by application of an antimicrobial solution such as isopropyl alcohol or chg. The material in this embodiment as well as the other described above can be absorbent and can have a small pore size that permits egress of gas or solutions without permitting entry of contamination material or organisms through the walls of the device.

FIGS. 19A-19H show an example embodiment of a single monolithic spongy absorbent permeable protective pod for protecting an intravascular (IV) joint; shown in an open configuration so the inner contents can be viewed and to demonstrate how access and assembly can be achieved using the longitudinal slit or gap in the sidewall that extends from end to end.

FIG. 19A shows an example embodiment of a shield attached to a male luer lock syringe that is to be assembled with removable needleless access valve with a male luer lock and an access end with female threads compatible with the male luer lock.

FIG. 19B shows an example embodiment of the shield from FIG. 19A surrounding and protecting the male luer lock syringe and needleless access valve from FIG. 19A within the shield during assembly.

FIG. 19C shows an example embodiment of the shield from FIG. 19A surrounding and protecting the male luer lock syringe and needleless access valve from FIG. 19A within the shield during assembly, just prior to assembly or just after disconnection. The shield has two ends so that the connectors can be joined and protected within the inner chamber of the shield while an IV line or other medical device such as syringe can extend beyond an end.

FIG. 19D shows an example embodiment of the needleless access valve from FIG. 19A with a cap. The capped end is exposed to the outside for access. The IV line is extending through the opposite end of the shield. By pushing or pulling on the IV line the capped end can be exposed or withdrawn into the protective shield. The cap protects a portion of the access end of the needless access valve but does not protect the IV joint where the female luer lock mates with the male luer lock of the removable needleless access valve.

FIG. 19E shows an example embodiment of the protective shield from FIG. 19A forming a plug within a test tube. The plug and the test tube form an inner chamber than encases the needleless access valve assembled to the female luer lock of the IV line, the needless access valve assembled to the cap with a male luer lock thread and a portion of the IV line. The test tube wall comprises a transparent wall so the contents within can be assessed, such as whether the inner chamber is empty, what device is enclosed, is there are visible leakage or is there any solution in the inner chamber. The test tube has a smaller diameter than the sponge in the free relaxed position and when the plug is inserted the test tube compress the sponge wall to close the wall gap. The sponge forms an absorbent permeable compressible circumferential wall the contours that to the outer surfaces of the enclosed devices and the test tube forms a non absorbent impermeable rigid transparent circumferential wall. The inner chamber can contain an antimicrobial solution that would soak, clean, protect or sterilize the outer surfaces of the enclosed devices. This can be delivered by impregnated material such as an alcohol swab. The antimicrobial solution can escape the inner chamber by evaporation or wicking through the inner chamber permeable surface to reduce long term chemical stress on the enclosed components. In addition antimicrobial solution can be delivered through the permeable wall to reload during a prolonged storage or prior to disconnection of the devices.

FIG. 19F shows an example embodiment of the plug from FIG. 19E with IV line assembly removed from the protective storage of the inner chamber.

FIG. 19 G shows an example embodiment the end cap from FIG. 19F removed with the needleless access valve access end retracted within the collar of the spongy material.

FIG. 19 H shows an example embodiment of the absorbent collar from FIG. 19G gripping the outer surface of the male luer lock collar during assembly to guide the connector ends together within the protective sleeve formed which blocks touch contamination with the surrounding environment during the assembly.

FIG. 20A shows an example embodiment of a preassembled needleless access valve and IV line with a female luer lock. The devices are placed within a protective chamber with a soft permeable material on the inside and a more rigid protective impermeable and hinged shell on the outside.

FIG. 20B shows an example embodiment of the hinged shell from FIG. 20A with a bivalve configuration in a closed configuration to protect the IV joint which is assembled with the IV line exiting beyond the shell layer. Additionally an antimicrobial solution is shown which had been applied to the absorbent inner material so that the antimicrobial solution is delivered to the outer surfaces of the enclosed assembled IV joint and devices and the solution is retained there for some time.

FIG. 21A shows an example embodiment of another hinged shell. The flexible material has been modified with slits to provide a placement area that will form a depression along the longitudinal axis to more easily accommodate and contour with the surface of the inserted medical connector and devices variable width surfaces.

FIG. 21B shows an example embodiment of the hinged shell from FIG. 21A in a closed configuration.

FIG. 22A shows an example embodiment of a protective pod with an inner chamber formed within a body that can be monolithic and can have two exit openings formed by a spongy, compliant, flexible, absorbent or permeable material. The groove or the exit openings can be preformed to fit over the IV lines; or the inner chamber and exits can be formed as the material contours over the outer surfaces of at least one of the medical devices. The pod can consist essentially of a monolithic body with or without an antibacterial material. The device is shown in an open configuration with a separated gap to demonstrate the inner contents and the manner of assembly through the sidewall.

FIG. 22B shows an example embodiment of an open protective outer shell placed around a protective pod. The protective outer shell is impermeable and rigid and compresses the inner material s

FIG. 22C shows an example embodiment of the protective outer shell from FIG. 22B in a closed position, containing the protective pod and IV lines therein. The open gap in the side wall is closed.

FIG. 22D shows an example embodiment of the protective outer shell from FIG. 22B re-opened for disconnection of the IV connectors, after having been stored in the safe protective inner chamber of this embodiment.

FIG. 22E shows an example embodiment of the male luer lock from FIG. 22B disconnected and unprotected from the needleless access valve in the protected chamber.

FIG. 22F shows an example embodiment of the protective chamber from FIG. 22E after it has been re-closed, sealing the needleless access valve. In comparison, in FIG. 22F, there is a single IV exiting the chamber in comparison to FIG. 22C, which has two IV lines exiting. The wet inner surface of the transparent casing caused by the antimicrobial solution impregnated in the spongy material can be seen.

FIG. 23 shows an example embodiment of a tubular shield with two pre-formed open ends with an open side that surrounds two IV lines, a needless access valve and a female luer lock connector. There can be a more rigid outer shell that helps to compress, close and retain the inner material that is optional and is not assembled.

FIG. 24 shows an example embodiment of a tubular shield with two pre-formed open ends with an open side. There is a more rigid outer shell that helps to compress, close and retain the inner material.

FIG. 25A shows an example embodiment of a tubular shield, including a rigid outer shell and a protective chamber, that can have two closed or two pre-formed open ends with an open side.

FIG. 25B shows an example embodiment of the more rigid outer shell and protective chamber from FIG. 25A that helps to compress, close and retain the inner material, including an IV line, that is partially assembled.

FIG. 25C shows an example embodiment of the IV line exiting the side gap of the more rigid outer shell and protective outer chamber from FIG. 25A to demonstrate how assembly within the shield and outer shell or removal from the shield and outer shell might occur to protect the IV joint between the female luer lock of the IV line and the male luer lock of the needleless access valve shown. As illustrated, the outer surfaces of these devices and the IV joint can be protected by a circumferential shield without the hazards of disconnecting them. It is shown that this embodiment does not require wrapping or unwrapping a tape multiple times which can lead to tangling or dislodgement of a delicate IV line with these movements.

FIG. 25D shows an example embodiment of the single IV line exiting the pod from FIG. 25C.

FIG. 25E shows an example embodiment of a needleless access valve within the shield from FIG. 25C that surrounds the outer circumference of the device. It has been assembled within the shield which provided a protective shield during assembly. The side slit can assist with placement prior to connection and assist with visualization before during and after connector assembly.

FIG. 25F shows an example embodiment of the side slit from FIG. 25E which can also be closed before, during and after assembly and can be surrounded by a more rigid clamping shell material. As illustrated is the exit of two connected IV lines from opposite ends of the pod.

FIG. 25G shows an example embodiment of how the inner chamber from FIG. 25E can be reopened for access and removal of the assembled connectors without disconnecting the IV connectors. Likewise the IV connectors and IV lines can be placed within the slit without disconnecting them.

FIG. 26A shows an example embodiment of an impermeable plug surrounding an IV line attached to a needleless access valve.

FIG. 26B shows an example embodiment of how the plug from FIG. 26A can have a slit for placement of an IV line.

FIG. 26C shows an example embodiment of how the separate plug and a test tube from FIG. 26A can be positioned prior to finished assembly.

FIG. 26E shows an example embodiment of the formation of an inner chamber than encases the needleless access valve from FIG. 26A assembled to the female luer lock of the IV line and the needless access valve assembled to the cap with a male luer lock thread and a portion of the IV line. The test tube wall comprises a transparent wall so the contents within can be assessed, such as whether the inner chamber is empty, what device is enclosed, is there any visible leakage, is there any mist or solution in the inner chamber. The test tube has a relatively smaller inner diameter than the outer diameter of the plug when in a non compressed free state. Assembly creates a snug fitting and seal. The plug forms an impermeable compressible circumferential wall that contours to the outer surfaces of the enclosed IV line and the test tube forms a non absorbent impermeable rigid transparent circumferential wall.

FIG. 26F shows an example embodiment of the impermeable inner chamber from FIG. 26E retaining an antimicrobial solution such as the CHG from the labeled bottle. The devices within the inner chamber are protected by the walls of the inner chamber and the antibacterial solution during assembled storage.

FIG. 26G shows an example embodiment of the impermeable inner chamber from FIG. 26F retaining an antimicrobial solution such as the isopropyl alcohol from the labeled bottle. The devices within the inner chamber are protected by the walls of the inner chamber and the antibacterial solution which can soak outer exposed surfaces.

FIGS. 27A-27H show a schematic diagram of an example IV line guard. As shown it can include a single component or it can have an inner foam layer surrounded by another layer. The component can fit around a connector snugly and it can have antibacterial and/or absorbent properties. It creates a sealed chamber and can self close with a built-in latch. It can include a cylindrical outer shell, an inner protective chamber, and a hinge to close around the IV line elements.

FIGS. 28A-28F show a schematic diagram of a protective outer shell with a clip for closing to halves, a sponge-like protective chamber, and a plug to contain the IV line elements entering it, such as a bulb. FIGS. 28A-28F can protect the end of an IV line element, even if not connected to a mating connector, or it can function with two paired connectors.

### DESCRIPTION OF EXAMPLE EMBODIMENTS

In general, the present disclosure includes an intravascular access system joint comprising an intravascular (IV) access line having a flexible IV access tube with a distal portion entering into a patient and a proximal portion bonded at the proximal end to a female luer hub. The system includes a first IV device with a first hub connector having a first female luer connector with a minimum standard luer connector length. The first female luer connector is connected to a first male luer connector of a second IV device hub connector of a second IV medical device. The system can have an apparatus with a protective barrier that encircles the IV joint along the longitudinal axis of the first and second hubs to form a chamber enclosing the external joint space between the first medical device and second medical device.

The apparatus can also include, individually or in combination, one or more of the following elements and characteristics: a pod with a pod inner chamber; a pod structured and arranged to be removable without breaking the assembly; a pod inner chamber that encloses the IV joint and a needleless access valve with a male luer (or luer lock) and female luer (or luer lock) threads; a pod inner chamber that encloses at least two IV connector assemblies, each with a male luer and female luer connection; a pod with a port for a first IV tube; a pod with two ends and a first port for a first IV tube and a second port for a second IV tube located at opposite ends of the pod; a pod inner chamber that encloses two IV tubes; a pod inner chamber that encloses the IV joint between the IV line and a needless access valve with a male luer (or luer lock) and female luer (or luer lock) threads connected to a proximal IV line; a pod that comprises a foam wall with an absorbent inner surface circumferentially surrounding the IV joint along the longitudinal axis; a pod that comprises a hinge; and a pod that can be split open down the longitudinal axis for easy removal tangential to the longitudinal axis of the hub connectors.

The present disclosure can also include an apparatus for protecting a patient by providing a protection barrier for an IV access system joint of an IV access system, so that the IV access system and patient are better protected from environmental contamination and microbial ingress between access events or while maintained in a connected configuration. The IV access system joint can include an intravascular (IV) access line having a flexible IV access tube with a distal portion entering into a patient and a proximal portion bonded at the proximal end to a female luer hub. The apparatus can also include a first IV device with a first hub connector having a first female luer connector with a minimum standard luer connector length. The first female luer connector is connected to a first male luer connector of a second IV device hub connector of a second IV medical device. The apparatus can include a protective barrier that encircles the IV joint along the longitudinal axis of the first and second hubs to form a chamber sealing the external joint space between the first medical device and second medical device from external contamination.

Additional embodiments can include, individually or in combination, one of more of the following elements or characteristics: a pod with a pod inner chamber; a pod that is structured and arranged to be removable tangentially from the longitudinal axis without breaking the IV joint assembly; a pod inner chamber that encloses the IV joint and a needless access valve with a male luer lock and female luer threads; a pod inner chamber that encloses at least two IV connector assemblies, each with a male luer and female luer connection; a pod inner chamber that encloses two IV tubes; a pod inner chamber that encloses the IV joint between the IV line and a needleless access valve with a male luer lock and female luer threads connected to a proximal IV line; a pod that comprises a port for a first IV tube; a pod that comprises two opposite ends, and a first port for a first IV tube and a second port for a second IV tube located opposite each other at opposite ends of the pod; a pod inner chamber that has antimicrobial properties; and a pod that comprises an absorbent inner surface impregnated with an antimicrobial solution and a smooth non-absorbent outer surface.

The present disclosure can also relate to a system for protecting a patient by providing a protection barrier of connections in an IV access lines. The system can include an IV joint. The IV joint can include a line for intravascular (IV) access having a flexible IV access tube with a distal portion entering into a patient and a proximal portion bonded at the proximal end to a first female luer hub having a first female luer connector and connected to a distal male luer connector of a needleless access valve with a proximal female thread. The system can include a removable protective barrier forming a pod that begins distal to the IV joint and extends beyond the female threaded end of the needleless access valve, along the longitudinal axis of the first and second hubs. The pod can include a pod inner chamber that encapsulates the external joint space between the first medical device and second medical device and the needless access valve.

Additional embodiments can include, individually or in combination, one of more of the following elements or characteristics: a first medical device that is a central venous line (CVL) device; a pod that includes a resilient inner material surrounded and compressed by a rigid outer material; a pod that includes a side opening to split the pod open to facilitate its removal tangential to the longitudinal axis of the IV joint (without requiring disassembly of an assembled IV joint assembly) and the pod inner chamber encloses the IV joint to seal it from external contamination and comprises a resilient inner surface with antimicrobial properties; a pod inner chamber that spans IV joint and another connection surface; and a pod inner chamber that spans two IV tubes.

Further embodiments can include, individually or in combination, one or more of the following elements or characteristics: a circumferential collar that surrounds a male or female luer device and comprises an absorbent material. The collar can have a slit on the side to facilitate removal from a luer connector tangential to the longitudinal axis.

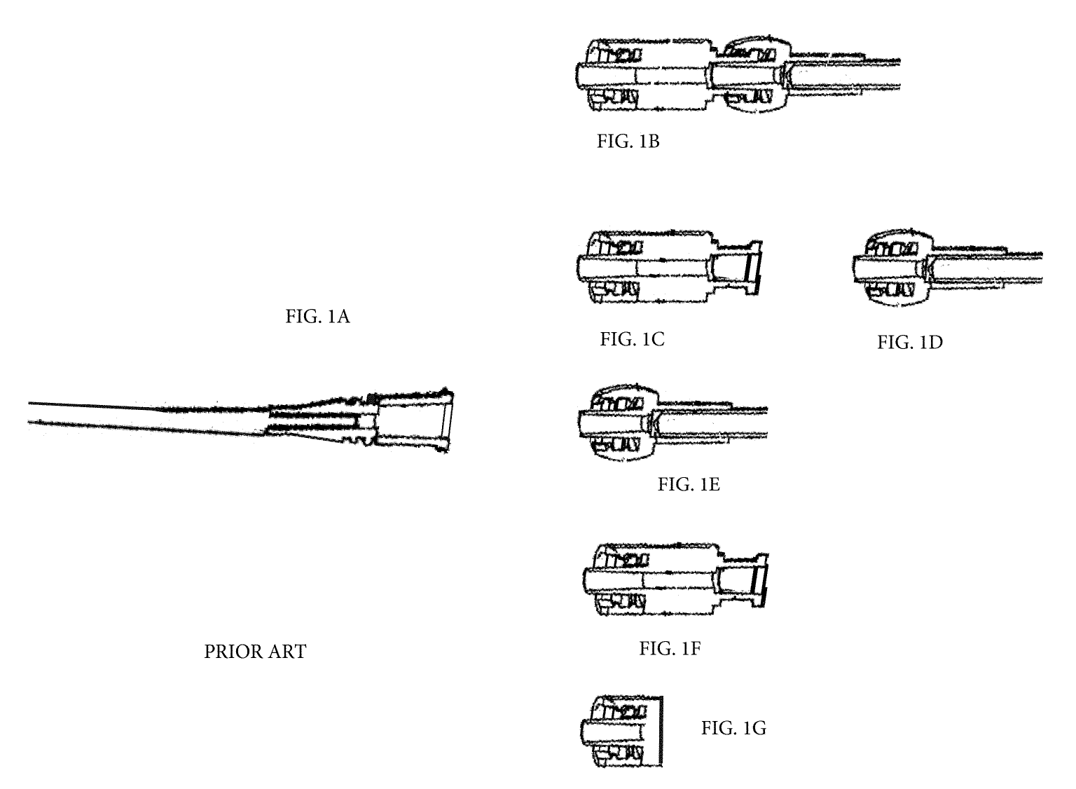

FIG. 1A is a schematic diagram of an example standard female luer lock connector and an intravenous line, such as a central line or peripheral IV line that goes to a patient.

FIG. 1B is a schematic diagram of an example IV valve such as a needleless access valve in connection with a male luer lock connector and an IV line.

FIGS. 1C and 1F are schematic diagrams of the example needleless access valve in FIG. 1B.

FIGS. 1D and 1E are schematic diagrams of the example male luer lock in FIG. 1B.

FIG. 1G is a schematic diagram of an example cap for a female luer lock.

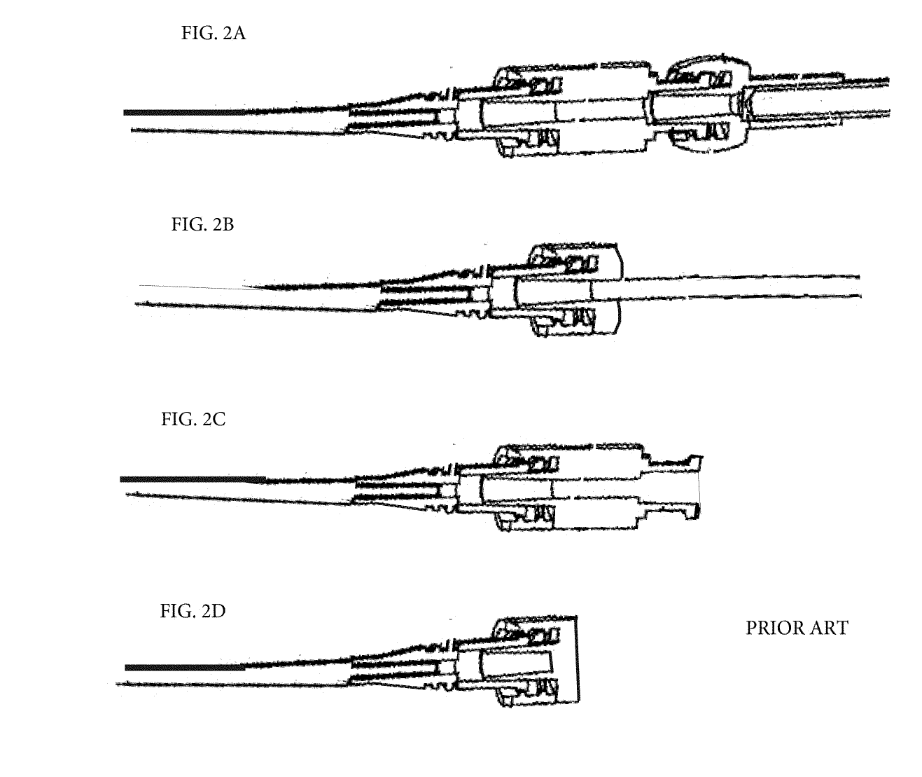

FIG. 2A is a schematic diagram of an example standard female luer lock connector attached to a first intravenous line, such as a central line or peripheral IV line that goes to a patient and assembled with a needleless access valve and assembled with a male luer lock in connection with a second IV line for supplying fluids. The joint between the female luer lock connector and the needleless access valve; as well as the joint between the needleless access valve and the male luer lock are unprotected from contamination.

FIG. 2B is a schematic diagram of an example standard female luer lock connector attached to a first intravenous line, such as a central line or peripheral IV line assembled with a male luer lock in connection with a second IV line for supplying fluids. The joint between the female luer lock connector and the male luer lock is unprotected from contamination.

FIG. 2C is a schematic diagram of an example standard female luer lock connector attached to a first intravenous line, such as a central line or peripheral IV line that goes to a patient and assembled with a needleless access valve. The joint between the female luer lock connector and the needleless access valve is unprotected from contamination.

FIG. 2D is a schematic diagram of an example standard female luer lock connector attached to first intravenous line, such as a central line or peripheral IV line that goes to a patient and assembled with a cap. The joint between the female luer lock connector and the cap is unprotected from contamination.

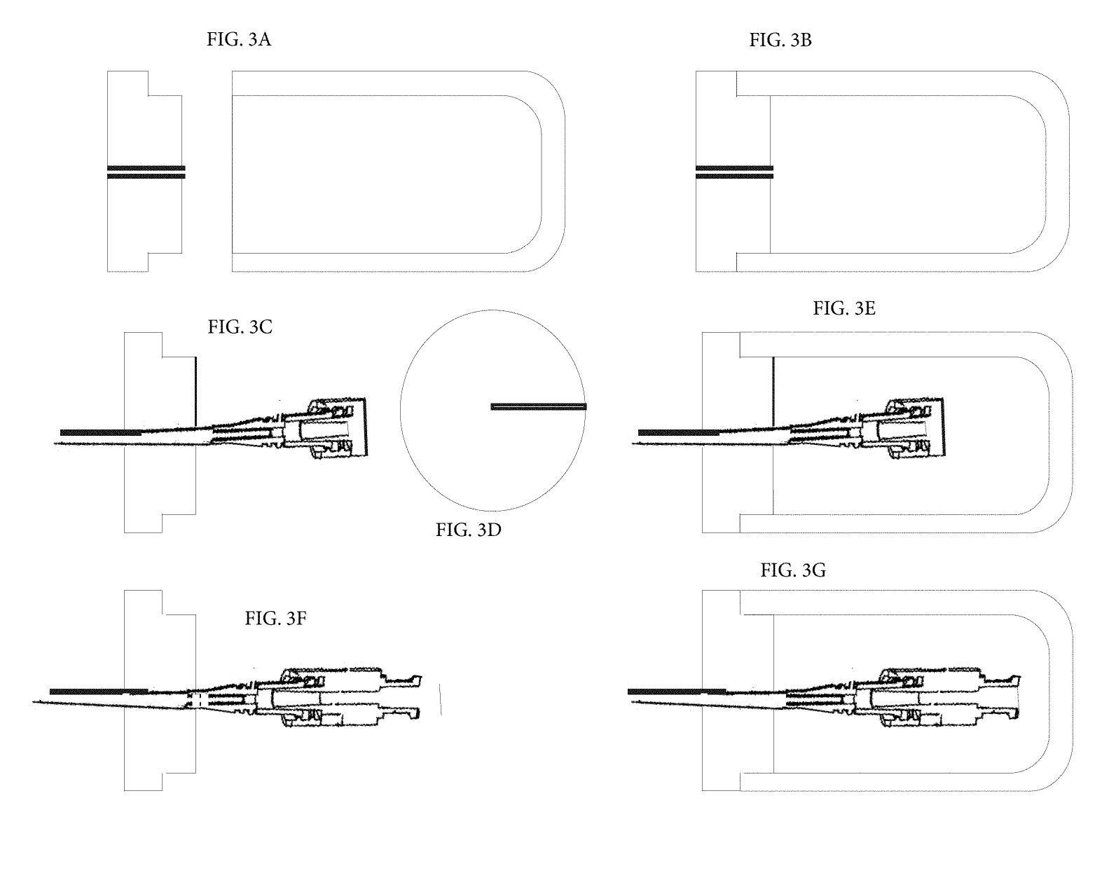

FIG. 3A is a schematic diagram of an example plug with a groove in the sidewall and a tube, such as a test tube, separated from each other.

FIG. 3B is a schematic diagram of the example plug and tube shown in FIG. 3A, with the plug inserted into the tube.

FIGS. 3C, 3E, 3F and 3G are schematic diagrams of the plug and tube shown in FIGS. 3A and 3B with an IV line and IV connector inserted within. The IV line can be insert plug and then into the tube while connected to an IV device such as cap or needless access valve. By placing the tube over the device with the IV connector and over the IV line surrounded by the plug, the tube and plug now provide a protective sealed chamber which can now protect the assembled IV devices from touch contamination and other types of contamination.

FIG. 3D is a top view of the plug shown in FIG. 3A, illustrating the groove. A cross-sectional view can be seen of the plug with the groove that can have walls touching with a potential space or can have a gap

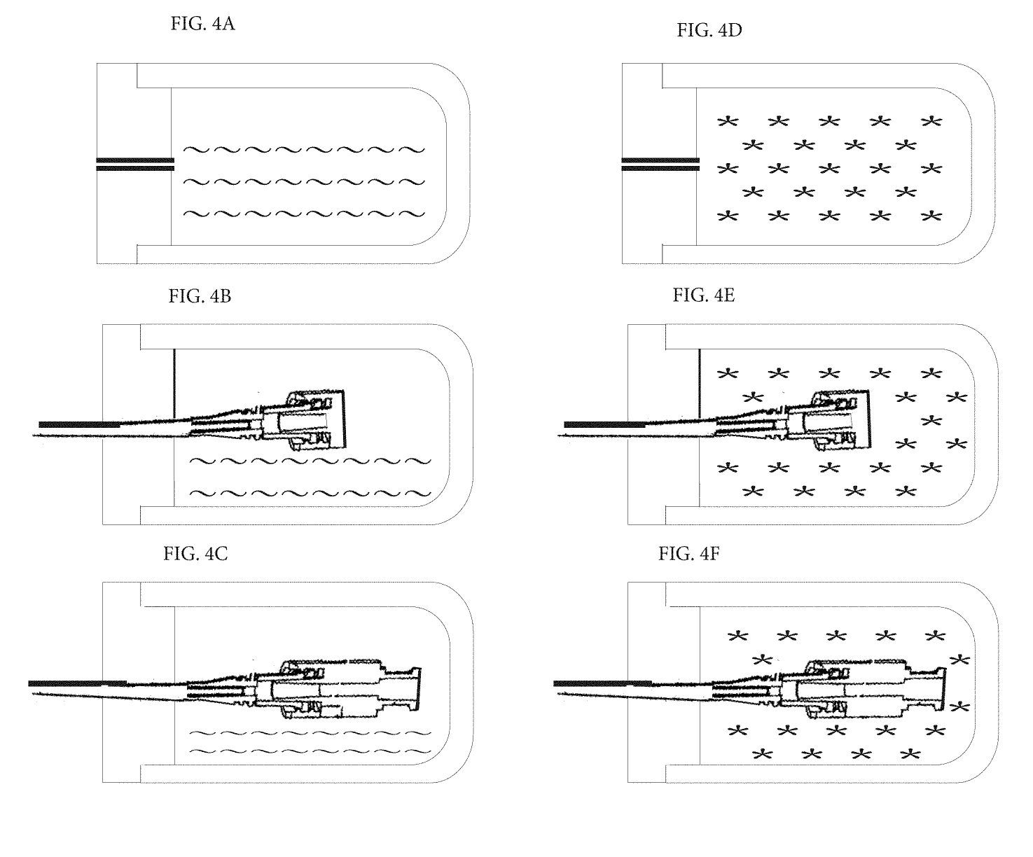

FIGS. 4A-4F are schematic diagrams of an example protective device with a tube and an inner chamber with an antimicrobial material such as antibacterial solution such as isopropyl alcohol or chlorhexidine gluconate (CHG) and an IV connector in FIGS. 4B-4F. There can be an evaporated or aerosolized gas within the tube as well.

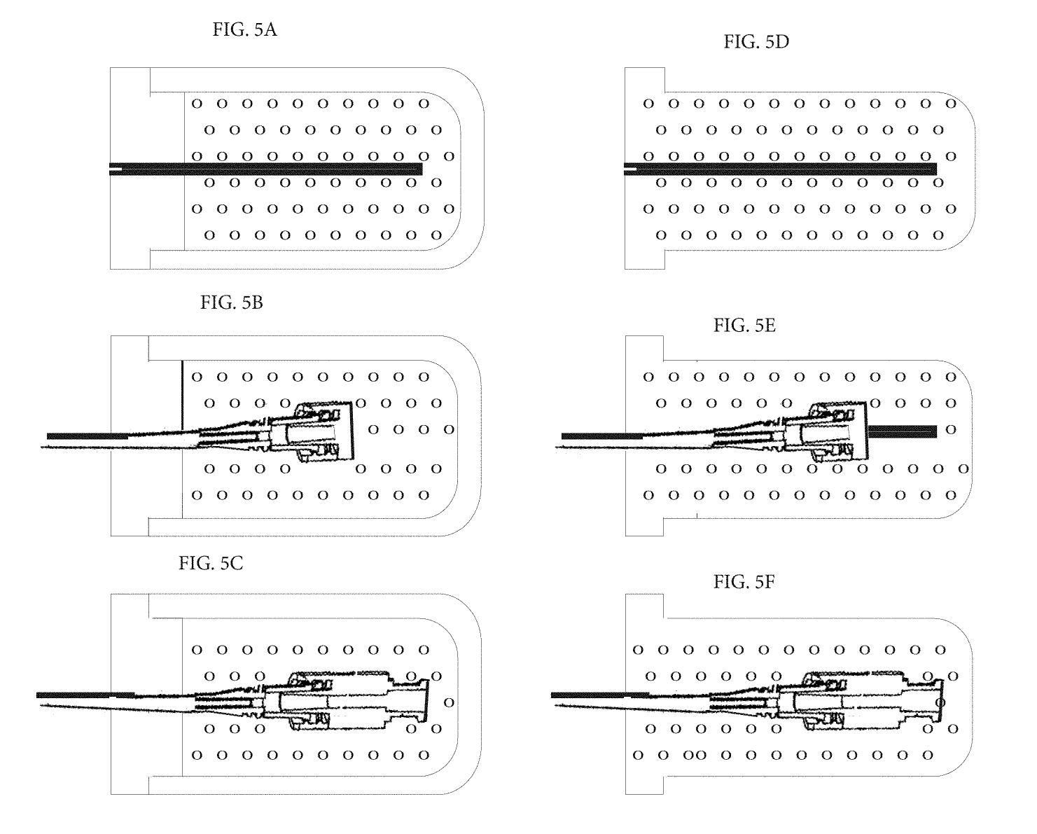

FIGS. 5A-5F are schematic diagrams of an example tube, a plug and protective chamber that is formed by a compliant material such as a spongy material. The material can be absorbent or it can be permeable. It can be non-absorbent and it can be non-permeable. There can be a groove in the side or center that permits insertion of the assembled medical devices for protection into an inner chamber. The chamber can be sealed on at least one end, or both ends. The sealed chamber can be permeable or impermeable. FIGS. 5B-5F show the protective chamber protecting IV connectors.

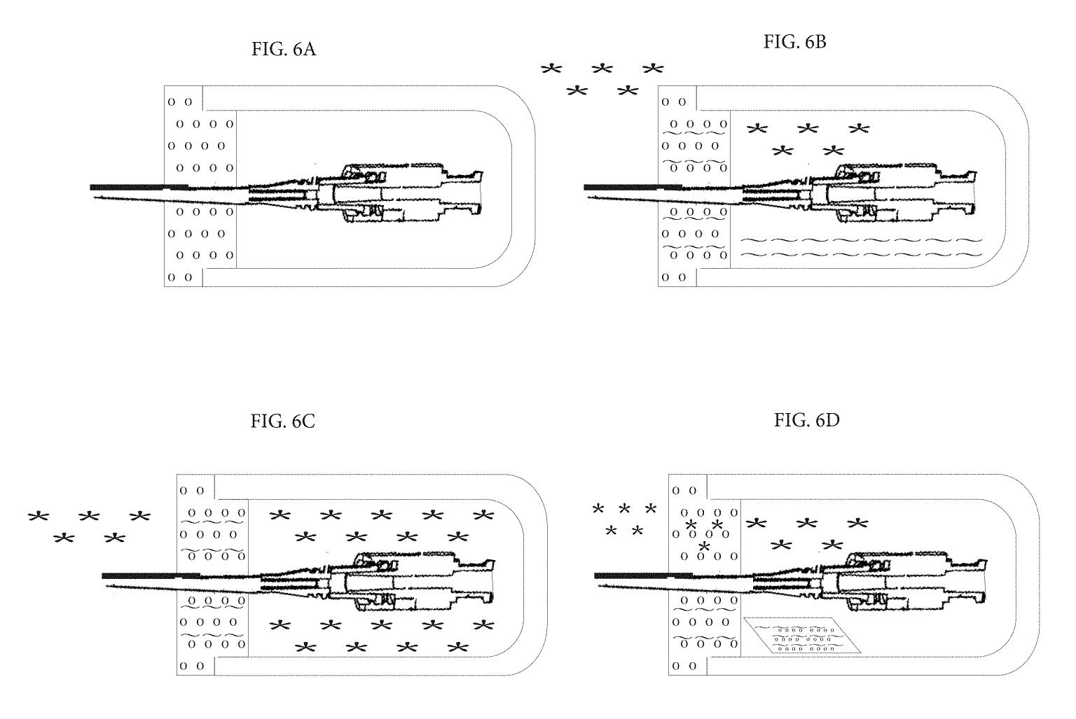

FIGS. 6A-6D are schematic diagrams of an example assembled medical device with the IV joint between the needleless access valve and the female luer lock protected within the inner chamber of the tube, such as the tube described above. The inner chamber can have an antimicrobial solution such as isopropyl alcohol, CHG or other agents. The inner chamber can contain an antibacterial aerosol or gas. The solution, aerosol, gas or other material can leave outer surfaces of the assembled medical device coated to prevent infection or can leave sterile. Outer surfaces of the devices, such as those within a male luer lock would have superior protection to the prior art since these areas can not be easily reached with standard cleaning techniques during maintenance, connection or disconnection. The gas can permeate through a permeable surface of the inner chamber such as a permeable plug. The chamber can have a transparent wall to assess the contents and the condition of the contents inside. The inner chamber can contain an impregnated antibacterial material such as an alcohol swab that can evaporated and kill bacteria within. The alcohol can be soaked up by an absorbent material or can escape as a gas through a permeable material to reduce the risk of the chemical compounds have any negative stress effects on the medical device plastics over a prolonged period of time.

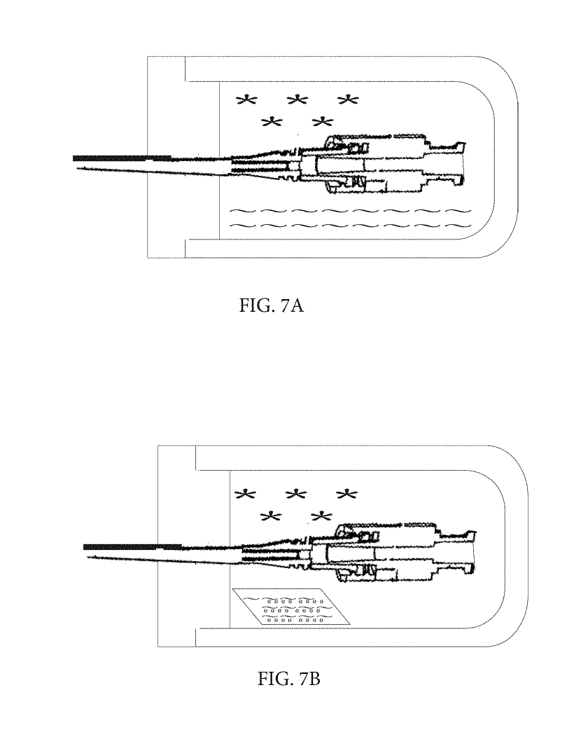

FIGS. 7A-7B show schematic diagrams of an example assembled medical device with the joint protected within the inner chamber within a tube such as the tube illustrated above. The inner chamber can have an antibacterial solution such as isopropyl alcohol, CHG or other agents. The inner chamber can contain an antibacterial aerosol or gas. The solution, aerosol, gas or other material can leave outer surfaces of the assembled medical device coated sterile. The solution or gas can be contained within the inner chamber which can be comprise impermeable walls that are resistant to the escape of the antibacterial solution. The inner chamber can contain an impregnated antibacterial material within the walls itself (FIG. 7A) or impregnated or soaked on separate material (FIG. 7B), e.g. an isopropyl alcohol swab, that can evaporated and kill bacteria within. The materials chosen can have protective properties that will reduce the risk of the chemical compounds having any negative stress effects on the medical device plastics over a prolonged period of time.

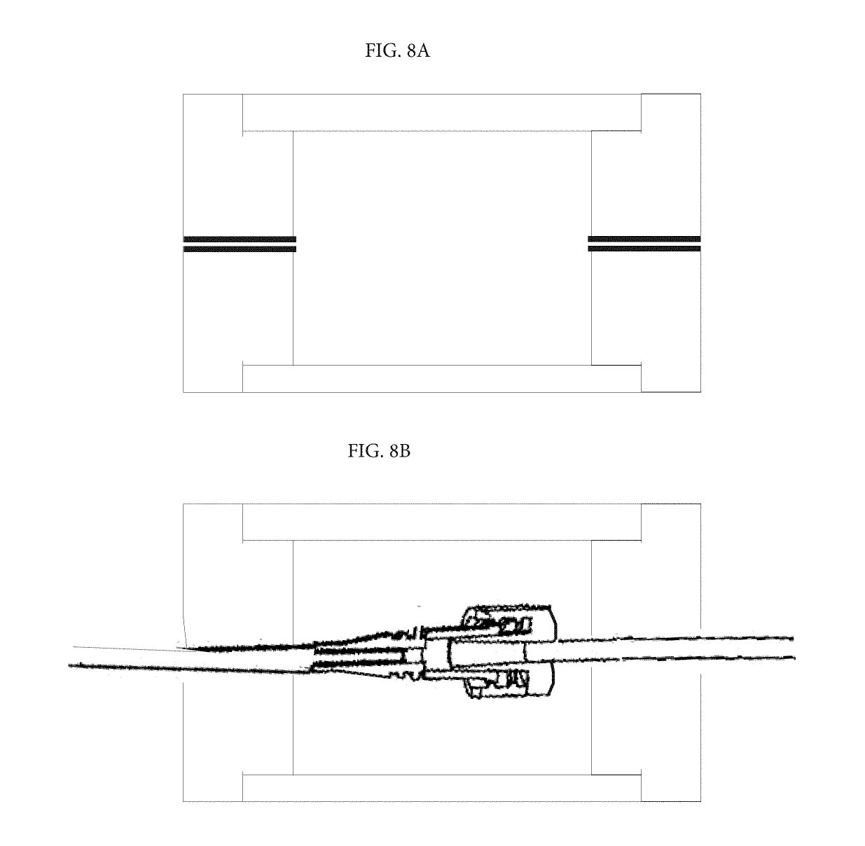

FIGS. 8A-8B are schematic diagrams of an example protective pod with an inner chamber can have two removable ends for use with a medical device assembly with a first and a second IV line at opposite ends.

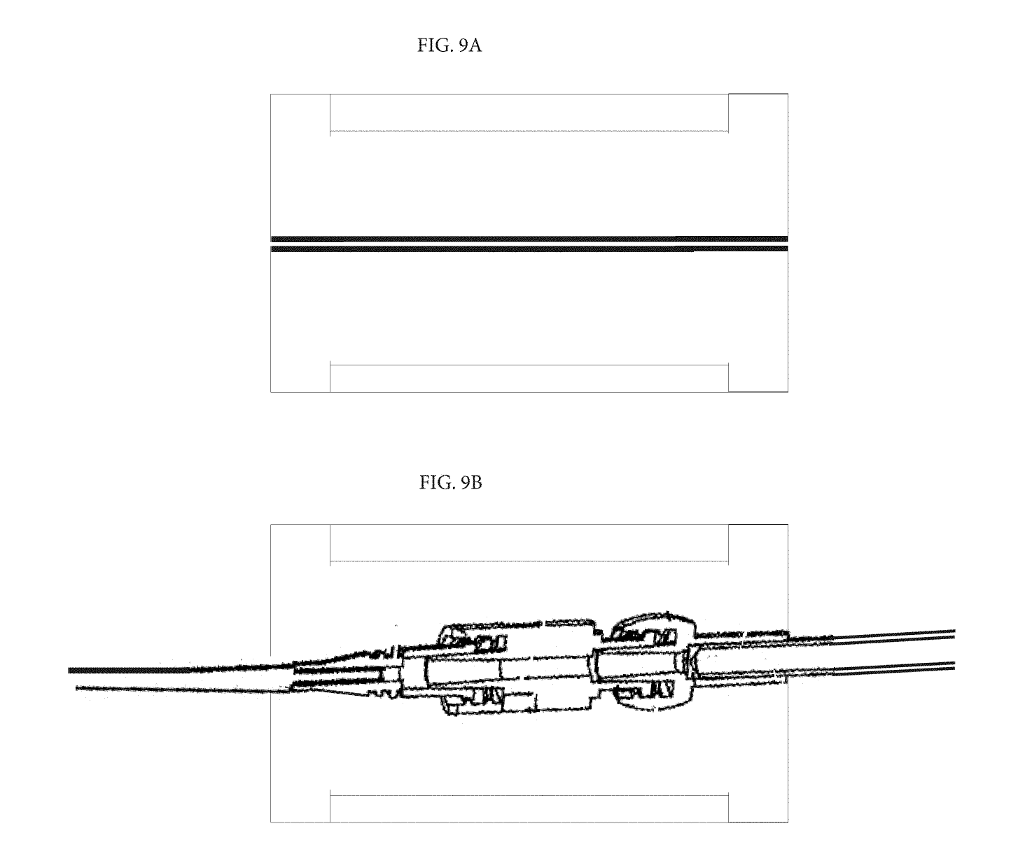

FIGS. 9A-9B are schematic diagrams of an example protective pod with an inner chamber formed within a body that can be monolithic and can have two exit openings. The groove and the exit openings can be pre-formed to fit over the IV line or the inner chamber and exits can be formed as the inner surfaces contour over the outer surfaces of at least one of the medical devices. There can be clamp or other restraining elements that maintains the walls of the inner chamber in their optimal positions such as shown in the middle portion of the outer wall surface. An IV line preassembled to at least one medical device can be placed through the groove and into the inner chamber which can be formed by material of the inner wall which would then be surrounding the contours of the assembled medical devices. The preassembled medical device can have both a first and a second IV line that can be a different positions, such as at opposite ends forming a substantially linear line assembly with two IV lines exiting different ports formed or preformed at opposite ends. This embodiment, like others described here, would permit a protective chamber with protective walls and at times protective substances to be applied to assembled medical devices without the risks associated with disconnection of those medical devices.

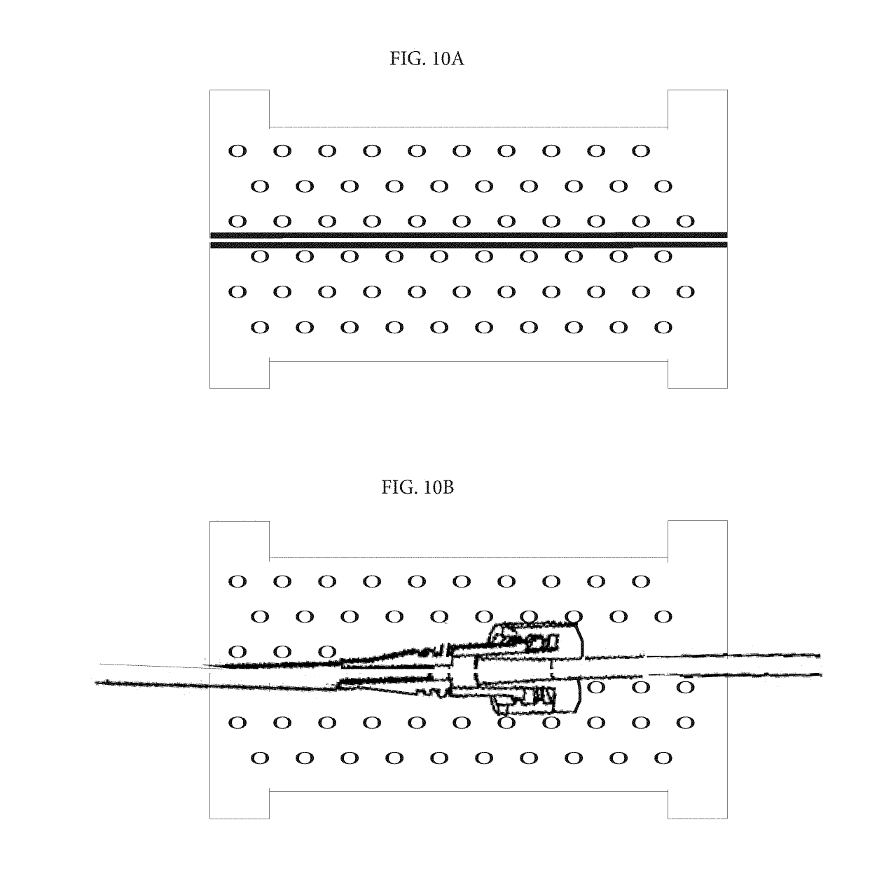

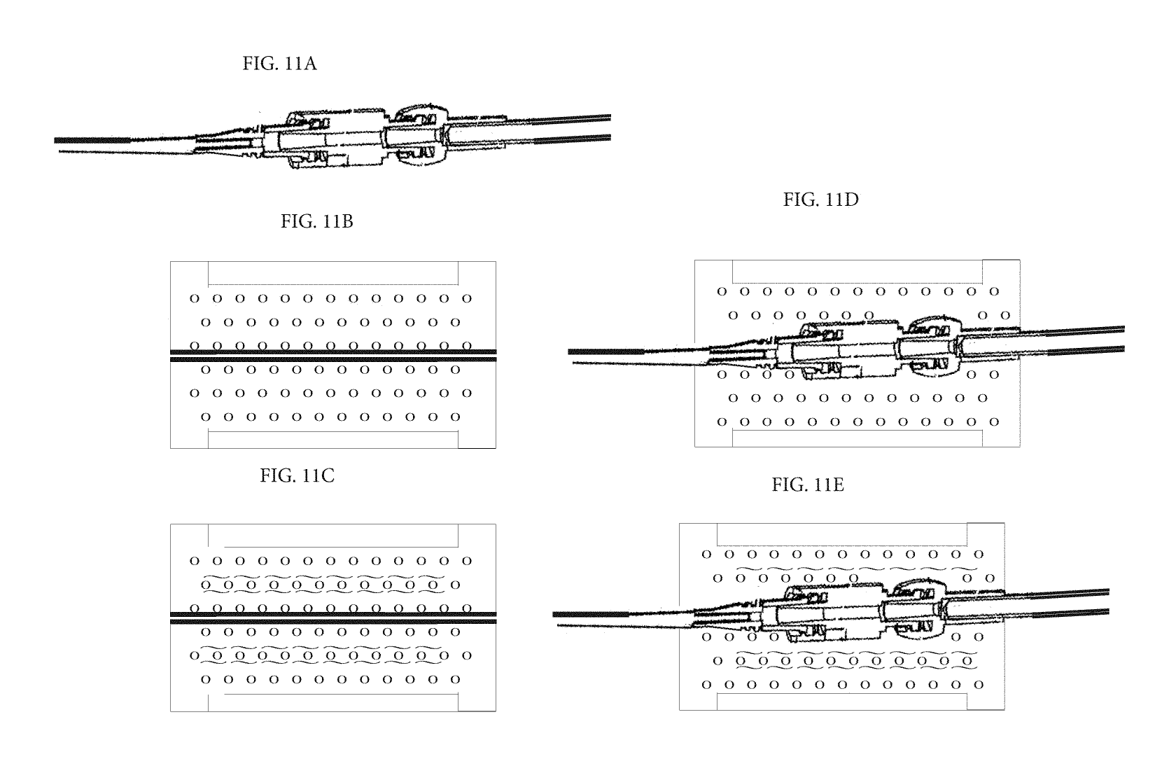

FIGS. 10A-10B are schematic diagrams of an example protective pod with an inner chamber formed within a body that can be monolithic and can have two exit openings formed by a spongy, compliant, flexible, absorbent or permeable material. The groove or the exit openings can be preformed to fit over the IV lines; or the inner chamber and exits can be formed as the material contours over the outer surfaces of at least one of the medical devices. The pod can consist essentially of a monolithic body with or without an antibacterial material.

FIGS. 11A-11E are schematic diagrams of an embodiment with an example clamp or other restraining elements that maintains the wall of the inner chamber in their optimal positions after an assembled medical device with at least a first IV line is placed within. Two IV line are shown pre-assembled. The device and other embodiment demonstrate that the assembled connectors do not need to be separated or have a free end in order to apply the protection device. This reduces the contamination risk associated with assembling free connector ends and the problems associated with leakage of the contents of a working IV line including waste, leakage, resetting flow monitors and inaccurate dosing of administer medications that are problems in the prior art. FIGS. 11B-11E also show embodiments with an impregnated solution. The solution can be pre-applied by the manufacturer or applied at the time of application about the IV assembly. It can be reapplied during a maintenance phase or just prior to removal. The solution can retained in place by an absorbent material or an antibacterial material can be impregnated into the material to protect against microbial contamination. The solution can evaporated leaving the inner chamber sterile or cleaner and the walls of the chamber can go from a moist material to dry material during this process. The material can include either super hydrophobic or super hydrophilic properties that would provide antimicrobial protection in either a wet or dry state. One such material is a material with “DAC” that is marketed by BSN Medical Inc. which binds bacteria when wet and prevents the bacteria from moving.

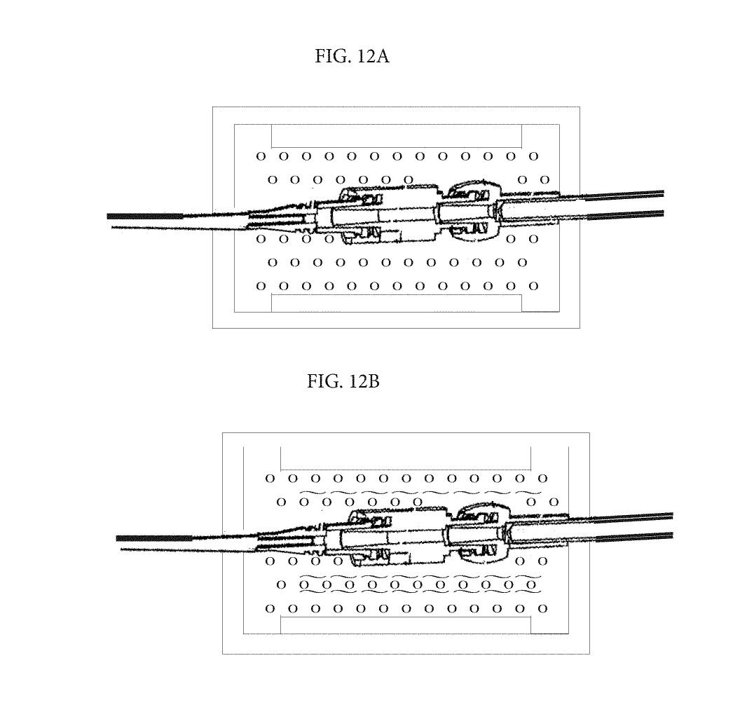

FIGS. 12A-12B are schematic diagrams of example embodiments with an outer protective layer that can be impermeable to protect against ingress of contamination and to retain an antimicrobial substance within the chamber. A protective layer can provide a seal around the assembly to reduce any entrance or exit through the pod outer surface to the inner channel of the assembled IV line or lines. While linear connections are shown, other devices, such a stopcocks or y-connectors (not shown) with multiple ports and non-linear connections might also be protected in the same way by the present invention. Embodiments of the device could easily be assembled and removed to assembled IV connectors, including assembled IV lines with IV connectors; e.g. much easier than a tape wound around the connections as has been done in the prior art to protect against disconnections. Embodiments of the present invention can also provide additional friction to the surface of the connected devices within the inner chamber and this can help prevent disconnection and the hazards associated with these events.

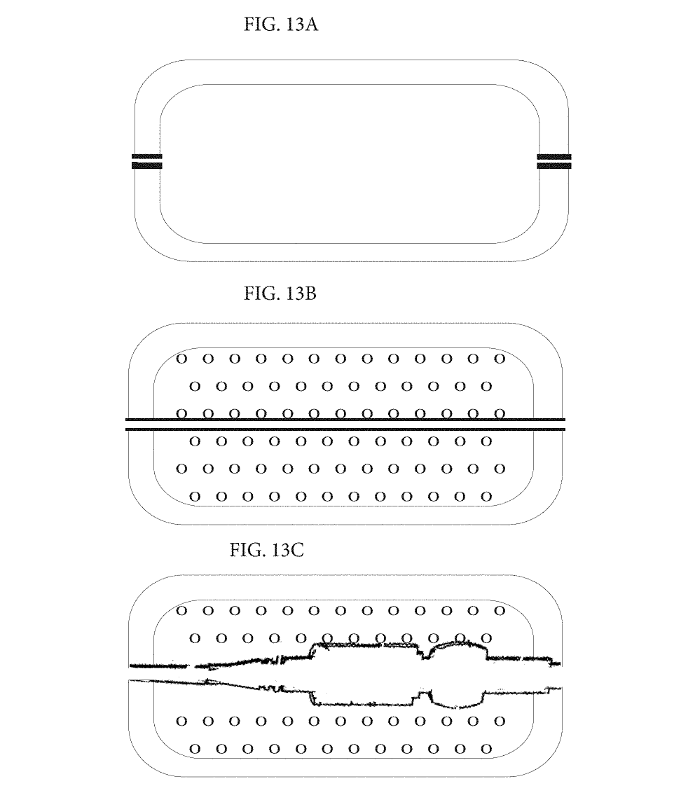

FIGS. 13A-13C are schematic diagrams of example different embodiments of the present invention. The apparatus can contain a hollow shell with preformed exit ports. The shell can be comprised of at least two valves or components that assemble together. (FIG. 13A). The valves can be hinged to assist with retention and proper placement. The shell can have a softer inner material to compress against a variety of medical devices with different contours. (FIG. 13B). Embodiments can have a pre-formed recess for a more precise positioning, fitting and engagement with the outer contours of two or more assembled IV devices and can fit over multiple device with at least two IV lines that exit out opposite ends of the protective inner chamber. (FIG. 13C). The material can have properties described elsewhere in this description such as the antimicrobial or permeability properties. Multiple embodiments are envisioned with a variety of recesses and restraints that could fit specific combinations of models of medical connectors for ideal positioning, sealing and retention.

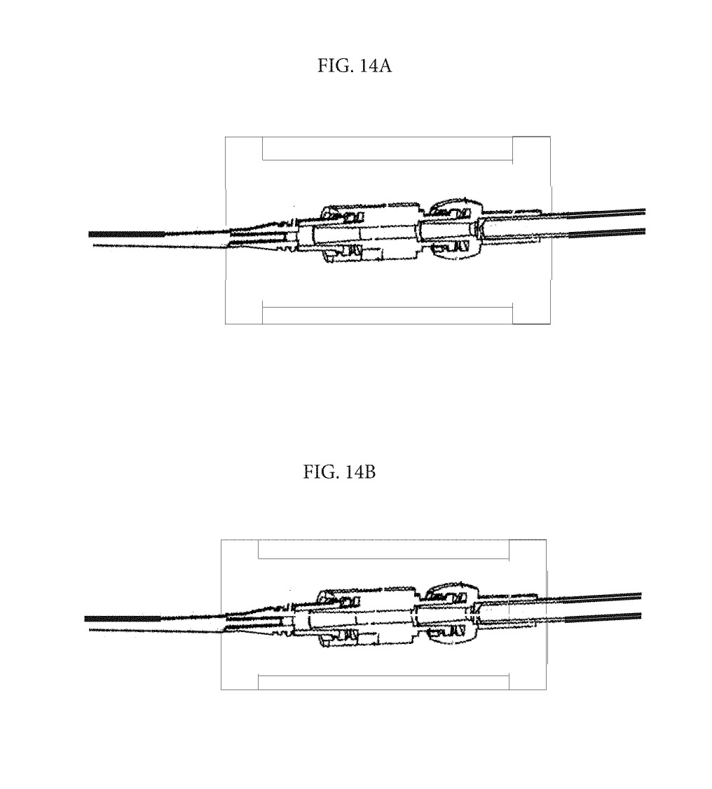

FIGS. 14A-14B are schematic diagrams of an example protective shell and inner wall with shrinking properties so that they can more tightly fit around the assembled IV device for better retention and sealing for improved protection of the devices. This might be achieved with a heat shrinkable material that is applied around the connected devices. Heat could be applied after application to shrink and seal the material and form a tighter more protective inner chamber. The shrink material can have antimicrobial properties that protect against contamination other than the physical barrier provided. The figure shows a cross sectional view of a wider pod (FIG. 14A) and a more narrow shrunken pod (FIG. 14B).

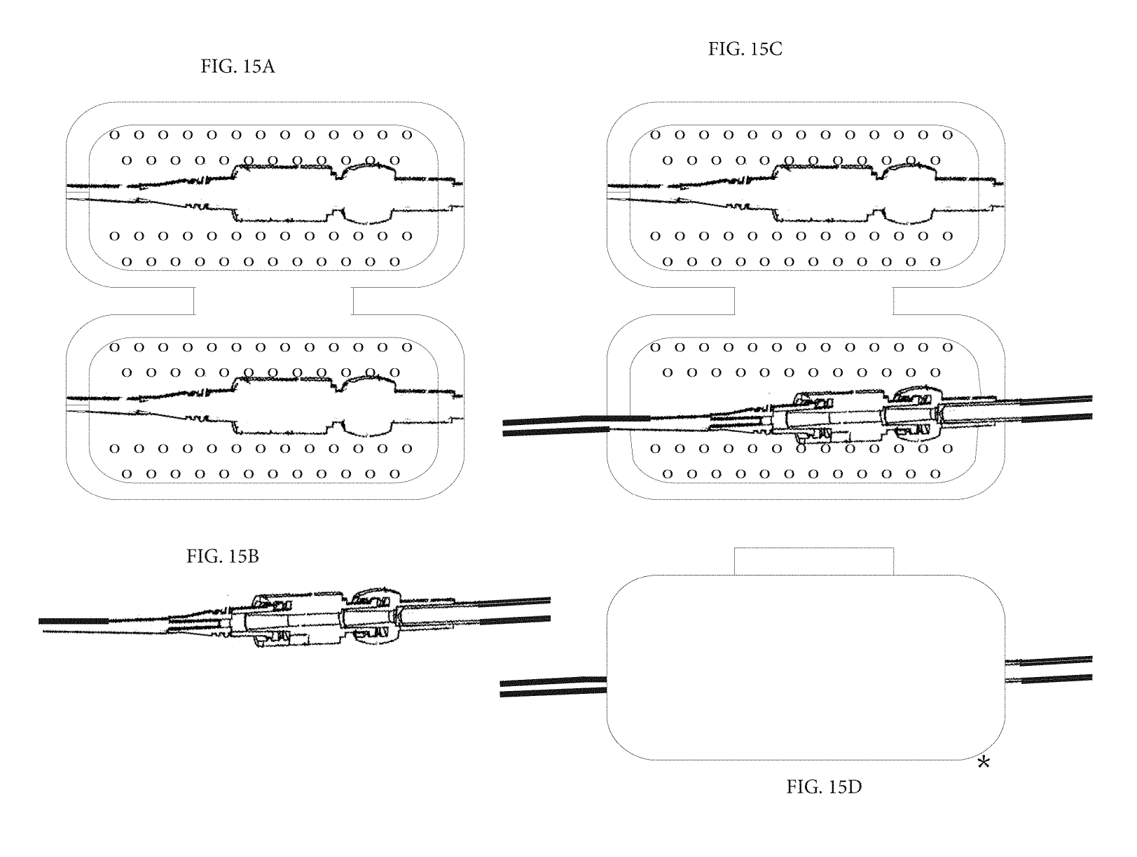

FIGS. 15A-15D are schematic diagrams of an example hinged bivalve shell with a soft material on the inside with at least one preformed recess for an assembled medical device. The assembled female luer lock, a needleless access valve and a male luer lock configuration with a supply IV line and a patient delivery IV line is placed within the pod though the open space along a longitudinal axis and when the bivalve shell is closed the assembled devices are protected by a sealed enclosure with an outer impermeable shell, an inner softer material impregnated with an antibacterial substance that is delivered to the outer surfaces of the medical device so that the inner material forming the chamber can provide surface antimicrobial protection and device retention. As shown in this embodiment and many others shown, the end openings can be smaller than the largest cross-sectional width of the connectors, smaller than the outer surface of male luer lock collar, smaller than the largest clearance of the inner threads of a standard ISO compliant male luer lock collar inner surface, smaller than the outer surface of a female luer lock threaded connector end, smaller than the distal tip of a male luer lock or smaller than the width of the IV tubing. In cases where an exit is smaller than a dimension listed, the opening can crimp the device or expand to accommodate it. For example, if the exit is smaller than the outer diameter of an IV tube, the material could clamp the outer surface of the IV tubing so it becomes smaller preferably without obstructing flow through the inner fluid channel and so the tubing is gripped well with a good outer seal over the tubing or the exit opening can expand to fit the size and contour of the outer wall of the IV tubing being stretched to form a good seal around the tubing.

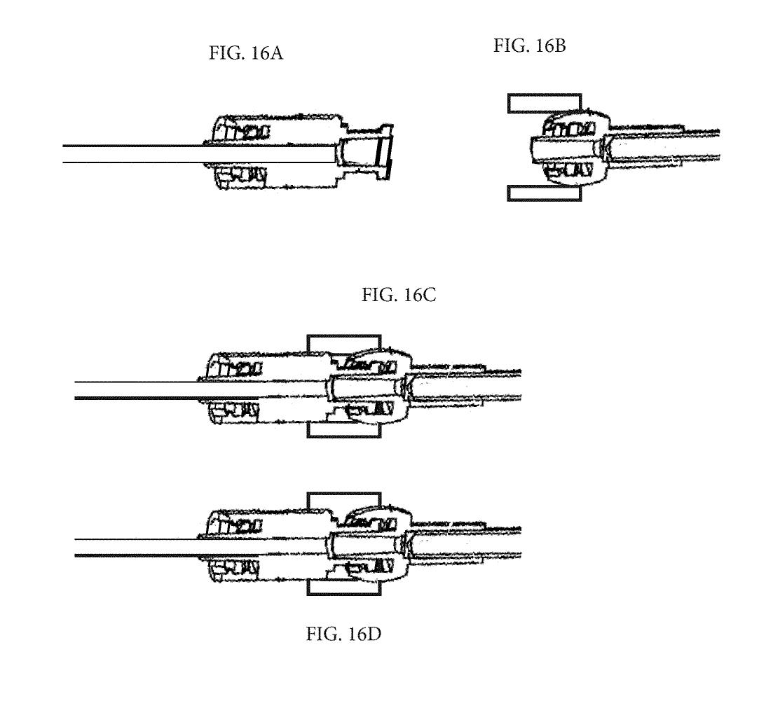

FIGS. 16A-16D are schematic diagrams of an example connector with male and female ends. The connector has at least one circumferential shield at either a male or a female end that creates a circumferential seal over at the ends of at least two connectors at the position where they are engaged. The shield can be applied during connection of the connectors and can assist with targeting of the connectors and protect from touch contamination during assembly of the male and female connector devices. The shield can have a slit in a sidewall along the longitudinal axis to allow the shield to be applied or removed after assembly or to provide for application of the shield from the side, rather than over the tip of the connector, to prevent disconnecting and opening the line. Application or removal from the side could also reduce the risk of accidental touch contamination of the critical connector end or tip surface with a non-sterile surface during manipulation.

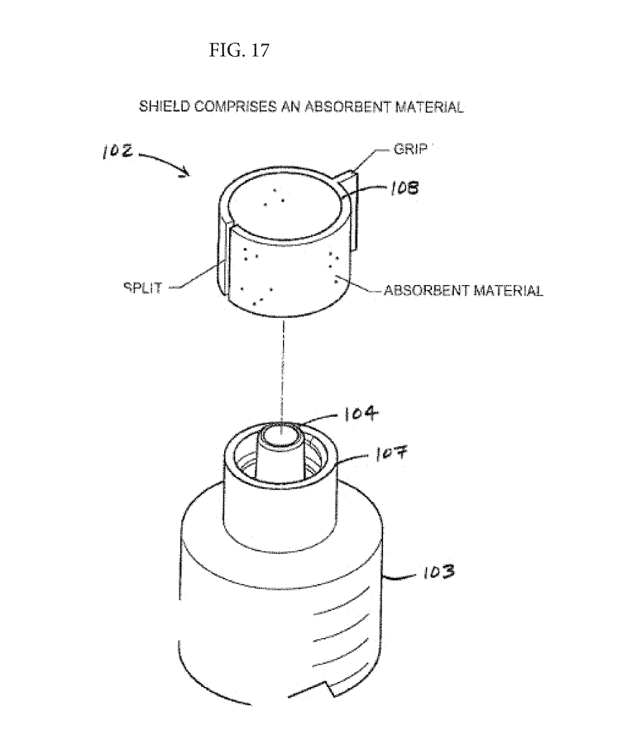

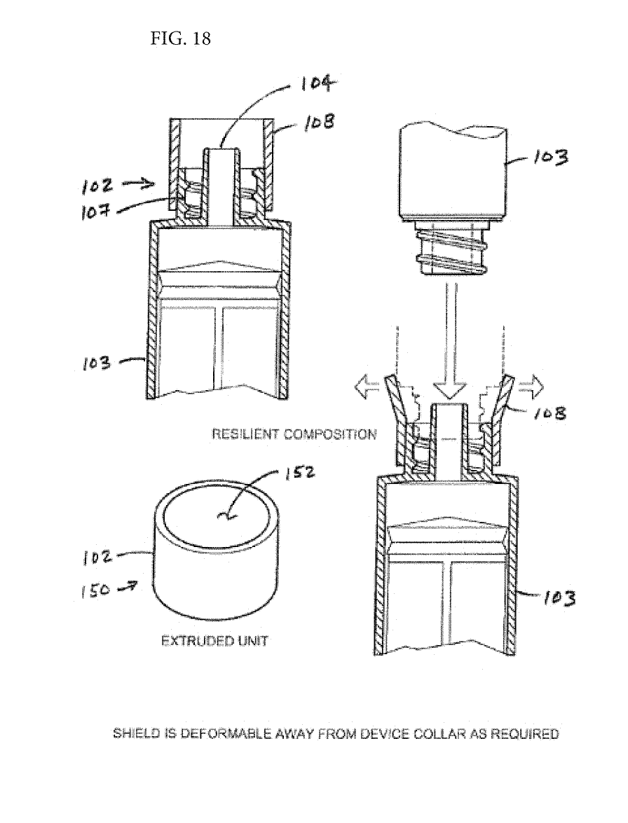

FIGS. 17-18 are schematic diagrams illustrating how a shield of the present invention can be delivered during assembly by one of the luer connectors, for example attached to the male connector end and delivered over the female luer connector end. There can be a pre-formed opening or the opening can be a potential space formed by the insertion of the connector on the receiving side. The end can be used for cleaning prior to insertion of the connector. There can be an open end of the shield with an inner clearance equal larger than the threads of a male luer lock collar or equal to or larger than the outer collar surface of a male luer lock collar or wide bodied needleless valve connector with a female connector with female luer lock threads that are compatible with a male luer lock. The shield can be removable from the male luer lock connector or the device with the male luer lock connector. The shield material can be absorbent to soak up blood or other material on assembly with a dripping IV hub or to soak up or deliver an antimicrobial substance to keep the shield and shielded connectors from becoming contaminated. If the shield were to get contaminated it can be constructed so that it can be removed from the side, e.g. if contaminated by blood after assembly of the connectors, and this could be accomplished without disconnection of the connectors. The shield with absorbent material can contain antibacterial properties and form a circumferential shield that can physically block contamination and can provide other anticontamination properties, e.g. by application of an antimicrobial solution such as isopropyl alcohol or CHG. The material in this embodiment as well as the other described above can be absorbent and can have a small pore size that permits egress of gas or solutions without permitting entry of contamination material or organisms through the walls of the device.

FIGS. 17-18 resemble FIGS. 37-39 from U.S. patent application Ser. No. 15/971,091, filed May 4, 2018, and hereby incorporated by reference for all intended purposes.

Under various circumstances, to improve the function of the shields and their compatibility and protective functions, inner clearance of these shields or the inner clearance of the internal chamber, can have inner dimensions less than or equal to the minimum diameter of the smallest cylinder that encompasses the outside surfaces of the external features of a fixed male luer lock collar of 8.800 mm; less than or equal to the nominal dimension of the smallest cylinder that encompasses the outside surfaces of the external features of a fixed male luer lock collar of 9.700 mm, less than or equal to the maximum dimension of the smallest cylinder that encompasses the outside surfaces of the external features of a fixed male luer lock collar of 11.500 mm to surround and grip the outer surfaces of certain parts of the devices such as a male luer lock collar.

Under various circumstances, to improve the function of the shields and their compatibility and protective functions, inner clearance of these shields or the inner clearances of the internal chamber with a continuous or discontinuous circumferential wall, can have an inner dimension greater than or equal to the minimum diameter of the smallest cylinder that encompasses the outside surfaces of the external features of a fixed male luer lock collar of 8.800 mm; greater than or equal to the nominal dimension of the smallest cylinder that encompasses the outside surfaces of the external features of a fixed male luer lock collar of 9.700 mm, or greater than or equal to the maximum dimension of the smallest cylinder that encompasses the outside surfaces of the external features of a fixed male luer lock collar of 11.500 mm to surround and outer surfaces of certain parts of the devices such as a male luer lock collar without contacting or obstructing the passage of the that portion of the device or to allow passage of compatible female connectors designed to fit within the outer collar dimensions or to provide a shield or inner chamber capable of sliding over or being placed circumferentially around a male luer lock collar of a particular dimension. These dimensions being referenced in Table B.3 and Figure B.3 from ISO 80369-7:2016(E), hereinafter incorporated by reference for all intended purposes.

Furthermore the shields would preferably extend greater than 2.1 mm from the distal end of the threaded collar and more preferably greater than 2.150 mm beyond the distal end of the threaded collar and or more preferably greater than 2.573 mm from the distal end of the collar; since the minimum, nominal and maximum dimensions for the projection of the tip of the male luer lock connector from the threaded collar in this recent iso standard have the dimensions of 2.100 mm, 2.150 mm and 2.573 mm respectively. Shields of these dimensions would ensure that the tip of the male luer connectors terminate within the shields rather than beyond for optimal protection during assembly, maintenance, temporary placement or longer term storage.

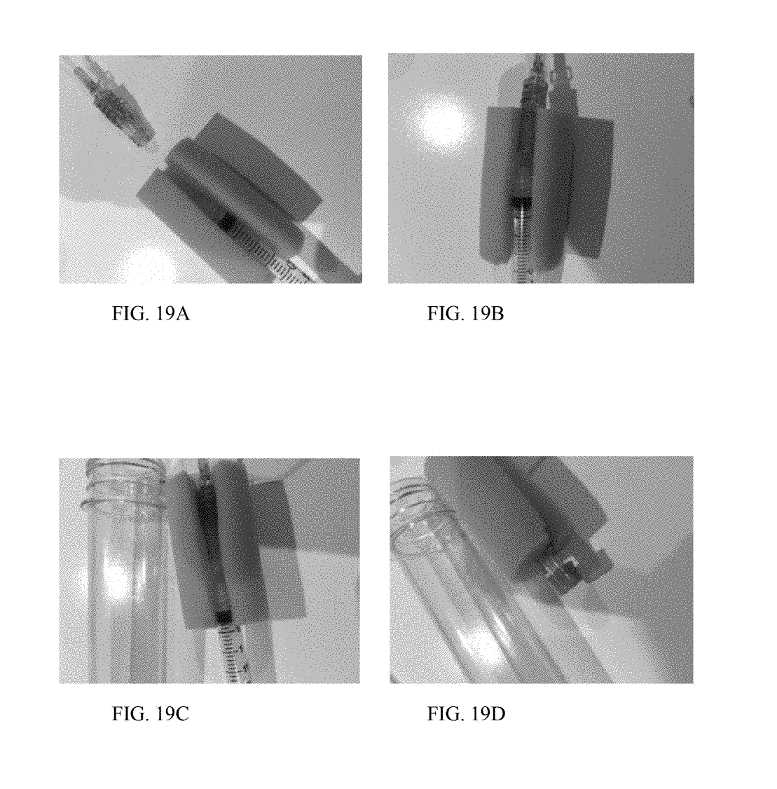

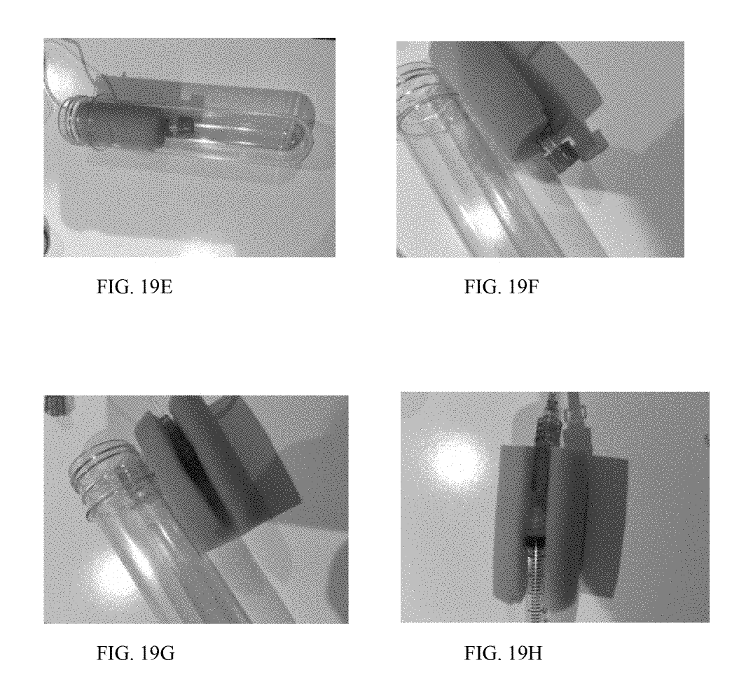

FIGS. 19A-19H illustrate an embodiment of an absorbent foam collar or sleeve around a Luer lock to help pre infection by absorbing material including infectious matter and by using as an absorbent layer impregnated with an antimicrobial solution, such as CHG, isopropyl alcohol or other appropriate substances. The foam collar protects the male and female luer locks during assembly and access. The collar is shown in an configuration with a slit or break in the circumferential wall to allow easier access and to the inside, to act as a spline, allow attachment and removal to around a male and female luer lock IV line joint without having to disconnect and open the system, but could also be an unbroken tube. The collar forms a seal below the base of the female needleless access assembly to protect this connection and forms a seal with an exterior barrier wall that forms a sealed internal chamber to protect IV system. As shown, the IV system is not actively being used in the sense that this needleless access valve is a dead end. It is stored in a sealed protective chamber as a physical barrier and has an antimicrobial solution within that provides additional protection for safety from infection during storage including prolonged storage when overgrowth is likely to occur. The protective chamber extends beyond the length of the needless access valve. It is wider than the widest portion of the needless access valve at some points and more narrow than the widest points at others. As shown two IV male and female luer lock thread assemblies are shown protected simultaneously. The chamber as shown is large enough to accommodate the male luer lock of the needleless access valve and the female luer lock of the IV tubing as well as the assembly of the female luer lock threads of the needless access valve and the male luer lock threads of the disinfection cap. This provides greater protection of the IV assemblies, IV line and the patient than the prior art of disinfection caps, especially for prolonged storage such as when central lines are used for chronic therapy. This also provides greater touch contamination protection during IV assembly and access. The protective chamber can be removed to provide the needleless access valve or the female luer lock in a protective sleeve, collar or spline that can have a wider radial clearance than that of a standard male luer lock collar and will help guide connection between the male and female connectors to prevent slippage and touch contamination. It also provides a protective collar against touch contamination during assembly and access. As shown the protective collar start on a male luer lock of a syringe and was transferred over an IV line assembly to protect the female luer lock for protective functions of the female luer lock, including protection of an assembly with a male luer lock at a later point. As shown there was a resilient inner layer surrounding the IV line at the base of the chamber with a port for the IV line. As shown there was a more rigid outer impermeable protective layer to protect the inner chamber and seal it. As shown the inner chamber had an antimicrobial solution such as isopropyl alcohol that could provide prolonged protection via liquid or vaporized contact within the chamber. As shown the protective chamber has single removable seal for a dead end system but as shown other embodiments of the invention can have two removable seals or plugs, or two ports to protect an assembled flowing line.

FIGS. 19A-19H show an example embodiment of a single monolithic spongy absorbent permeable protective pod for protecting an intravascular (IV) joint; shown in an open configuration so the inner contents can be viewed and to demonstrate how access and assembly can be achieved using the longitudinal slit or gap in the sidewall that extends from end to end.

FIG. 19A shows an example embodiment of a shield attached to a male luer lock syringe that is to be assembled with removable needleless access valve with a male luer lock and an access end with female threads compatible with the male luer lock.

FIG. 19B shows an example embodiment of the shield from FIG. 19A surrounding and protecting the male luer lock syringe and needleless access valve from FIG. 19A within the shield during assembly.

FIG. 19C shows an example embodiment of the shield from FIG. 19A surrounding and protecting the male luer lock syringe and needleless access valve from FIG. 19A within the shield during assembly, just prior to assembly or just after disconnection. The shield has two ends so that the connectors can be joined and protected within the inner chamber of the shield while an IV line or other medical device such as syringe can extend beyond an end.

FIG. 19D shows an example embodiment of the needleless access valve from FIG. 19A with a cap. The capped end is exposed to the outside for access. The IV line is extending through the opposite end of the shield. By pushing or pulling on the IV line the capped end can be exposed or withdrawn into the protective shield. The cap protects a portion of the access end of the needless access valve but does not protect the IV joint where the female luer lock mates with the male luer lock of the removable needleless access valve.

FIG. 19E shows an example embodiment of the protective shield from FIG. 19A forming a plug within a test tube. The plug and the test tube form an inner chamber than encases the needleless access valve assembled to the female luer lock of the iv line, the needless access valve assembled to the cap with a male luer lock thread and a portion of the iv line. The test tube wall comprises a transparent wall so the contents within can be assessed, such as whether the inner chamber is empty, what device is enclosed, is there are visible leakage or is there any solution in the inner chamber. The test tube has a smaller diameter than the sponge in the free relaxed position and when the plug is inserted the test tube compress the sponge wall to close the wall gap. The sponge forms an absorbent permeable compressible circumferential wall the contours that to the outer surfaces of the enclosed devices and the test tube forms a non absorbent impermeable rigid transparent circumferential wall. The inner chamber can contain an antimicrobial solution that would soak, clean, protect or sterilize the outer surfaces of the enclosed devices. This can be delivered by impregnated material such as an alcohol swab. The antimicrobial solution can escape the inner chamber by evaporation or wicking through the inner chamber permeable surface to reduce long term chemical stress on the enclosed components. In addition antimicrobial solution can be delivered through the permeable wall to reload during a prolonged storage or prior to disconnection of the devices.

FIG. 19F shows an example embodiment of the plug from FIG. 19E with IV line assembly removed from the protective storage of the inner chamber.

FIG. 19 G shows an example embodiment the end cap from FIG. 19F removed with the needleless access valve access end retracted within the collar of the spongy material.

FIG. 19 H shows an example embodiment of the absorbent collar from FIG. 19G gripping the outer surface of the male luer lock collar during assembly to guide the connector ends together within the protective sleeve formed which blocks touch contamination with the surrounding environment during the assembly.

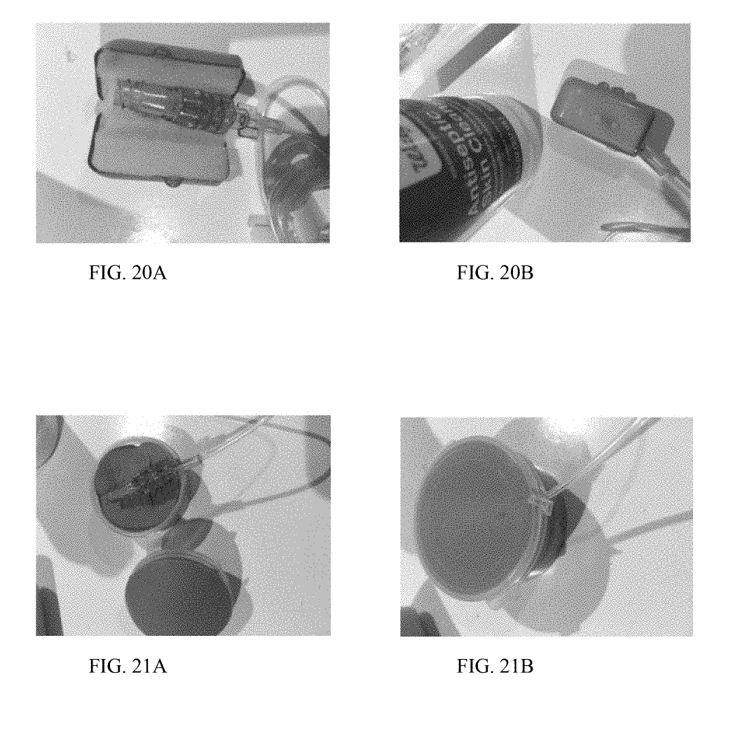

FIGS. 20B-20B illustrate a protective chamber with a resilient foam impregnated with an antimicrobial solution of Chlorhexidine Gluconate inner layer and impermeable more rigid outer layer that compresses the inner layer to increase and provide more uniform contact with the enclosed needleless access valve and IV line assembly. The entire needleless access valve is enclosed in the casing, and two female luer thread connectors are protected from two different components.

The circular casing shows a clam shell design which is hinged. The hinge reduces loose parts and guides the two casing halves. The casing surrounds an IV line and as shown it surrounds an IV line with a needleless access valve. The casing can protect during assembly of components, during storage of dead end components. It can protect during active administration of fluid through the needleless but as shown above the needleless access valve is in a dead end configuration.

As shown the needleless access valve is an active flow configuration. The connectors remain connected when a protective layer assembled around it. The layer has a break in a tubular design to allow this assembly to occur. The inner foam layer can be impregnated with antimicrobial solution. The rigid outer impermeable casing surrounds the inner layer to compress it against the IV components. The casing can have port to fit around the tubing to prevent significant compression of the IV tubing which might interrupt flow through the IV tubing. Additionally, as shown the protective collar and casing can be opened and used for protection of either male or female IV components during IV assembly and access vide dead end protection to a disconnected IV line. Tubing can be foam including an impregnated foam and can be used alone as a single layer or as a double layer.

FIG. 20A shows an example embodiment of a preassembled needleless access valve and IV line with a female luer lock. The devices are placed within a protective chamber with a soft permeable material on the inside and a more rigid protective impermeable and hinged shell on the outside.

FIG. 20B shows an example embodiment of the hinged shell from FIG. 20A with a bivalve configuration in a closed configuration to protect the IV joint which is assembled with the IV line exiting beyond the shell layer. Additionally an antimicrobial solution is shown which had been applied to the absorbent inner material so that the antimicrobial solution is delivered to the outer surfaces of the enclosed assembled IV joint and devices and the solution is retained there for some time.

FIGS. 21A-21B illustrate embodiments that can be a single layer or double layers that provide improved assembly and access. The slits in the double tubes can be offset to ensure complete circumferential protection. The outer layer can compress the inner layer. As shown the foam tube has an opening in the relaxed positioned but when encircled by the more rigid tubing the opening is closed at the free end and sealed against the tubing at the other end. The tubing can wrap around the line for easier assembly and protection, without disassembling the IV line. The single or double protective components do not require revolutions around the line like when using a tape, so that they are less cumbersome to apply and remove, are more likely to dislodge or contaminate IV lives.

Specifically, FIG. 21A shows an example embodiment of another hinged shell. The flexible material has been modified with slits to provide a placement area that will form a depression along the longitudinal axis to more easily accommodate and contour with the surface of the inserted medical connector and devices variable width surfaces. FIG. 21B shows an example embodiment of the hinged shell from FIG. 21A in a closed configuration.

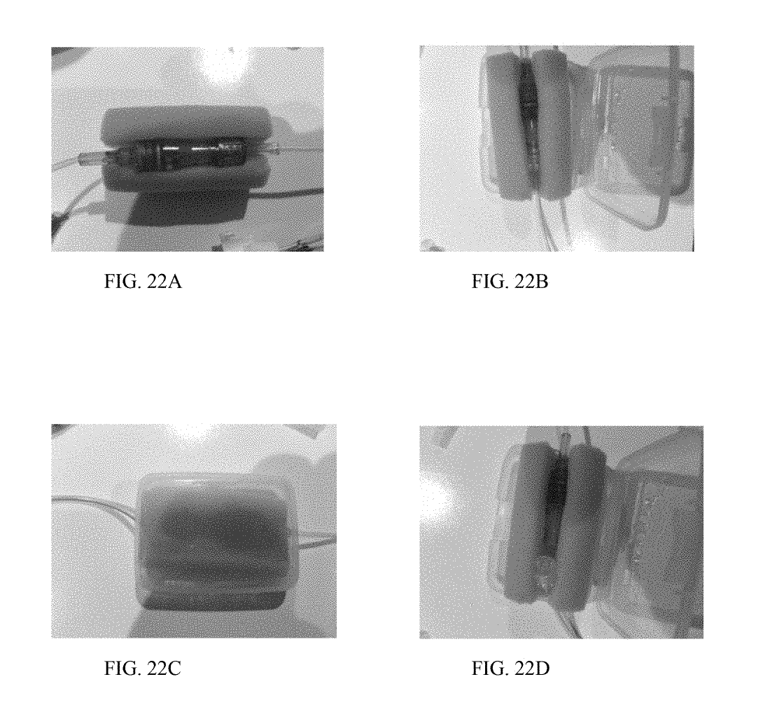

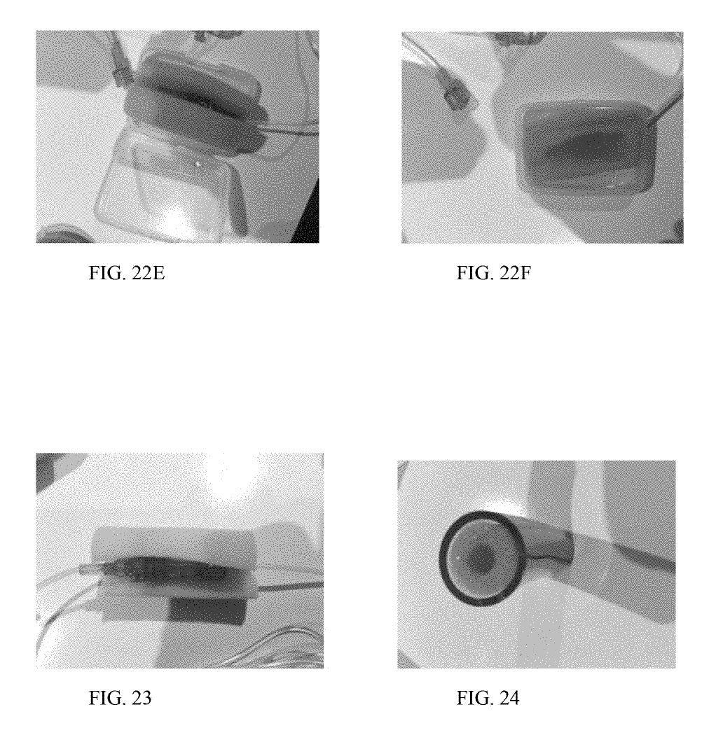

FIGS. 22A-22F illustrate an IV line that is inserted and encircled by a protective component along the longitudinal axis. The IV component is then inserted into a protective chamber. The protective chamber seals the IV component from contamination. This provides greater surface protection than disinfection caps marketed which on provide limited surface area contact and only protect the end of the needleless access valve shown, whereas the protective chamber shown protects the entire outer surface area of the needleless access valve. This helps to maintain its cleanliness during use and after being removed from the manufacturer's sterile packaging.

The chamber can also seal in an antimicrobial solution that protects the IV component. As shown the needleless access valve is bathed in CHG and isopropyl alcohol.

FIG. 22A shows an example embodiment of a protective pod with an inner chamber formed within a body that can be monolithic and can have two exit openings formed by a spongy, compliant, flexible, absorbent or permeable material. The groove or the exit openings can be preformed to fit over the IV lines; or the inner chamber and exits can be formed as the material contours over the outer surfaces of at least one of the medical devices. The pod can consist essentially of a monolithic body with or without an antibacterial material. The device is shown in an open configuration with a separated gap to demonstrate the inner contents and the manner of assembly through the sidewall.

FIG. 22B shows an example embodiment of an open protective outer shell placed around a protective pod. The protective outer shell is impermeable and rigid and compresses the inner material s

FIG. 22C shows an example embodiment of the protective outer shell from FIG. 22B in a closed position, containing the protective pod and IV lines therein. The open gap in the side wall is closed.

FIG. 22D shows an example embodiment of the protective outer shell from FIG. 22B re-opened for disconnection of the IV connectors, after having been stored in the safe protective inner chamber of this embodiment.

FIG. 22E shows an example embodiment of the male luer lock from FIG. 22B disconnected and unprotected from the needleless access valve in the protected chamber.

FIG. 22F shows an example embodiment of the protective chamber from FIG. 22E after it has been re-closed, sealing the needleless access valve. In comparison, in FIG. 22F, there is a single IV exiting the chamber in comparison to FIG. 22C, which has two IV lines exiting. The wet inner surface of the transparent casing caused by the antimicrobial solution impregnated in the spongy material can be seen.

FIG. 23 shows an example embodiment of a tubular shield with two pre-formed open ends with an open side that surrounds two IV lines, a needless access valve and a female luer lock connector. There can be a more rigid outer shell that helps to compress, close and retain the inner material that is optional and is not assembled.

FIG. 24 shows an example embodiment of a tubular shield with two pre-formed open ends with an open side. There is a more rigid outer shell that helps to compress, close and retain the inner material.

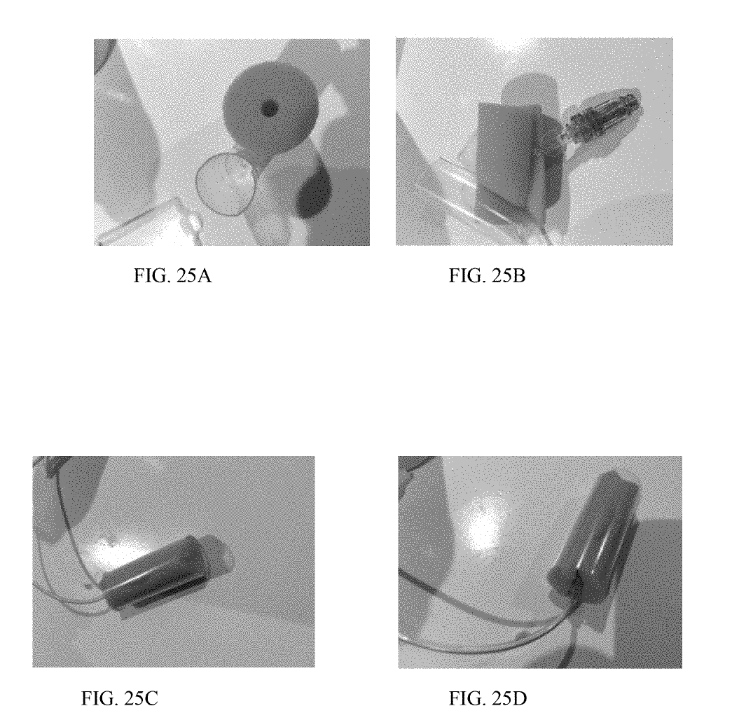

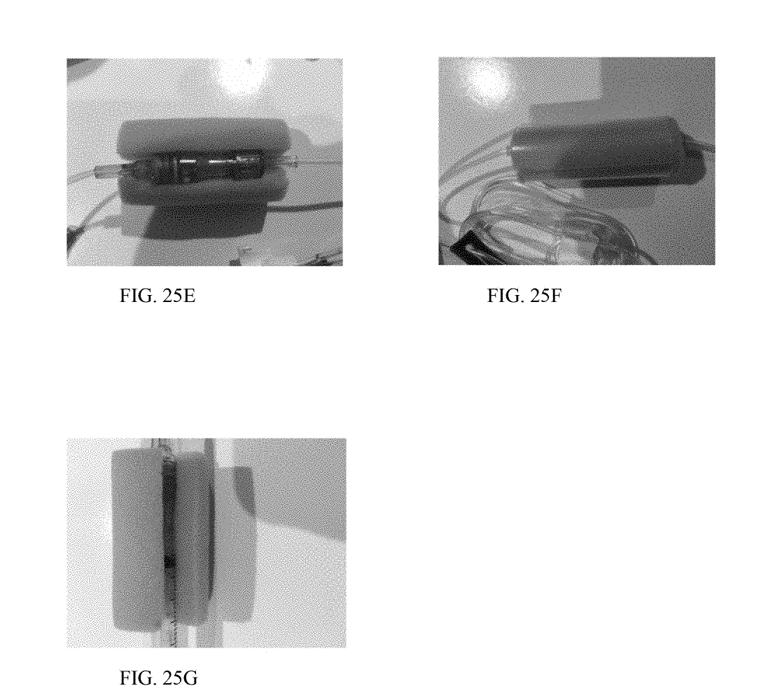

FIG. 25A shows an example embodiment of a tubular shield, including a rigid outer shell and a protective chamber, that can have two closed or two pre-formed open ends with an open side.

FIG. 25B shows an example embodiment of the more rigid outer shell and protective chamber from FIG. 25A that helps to compress, close and retain the inner material, including an IV line, that is partially assembled.

FIG. 25C shows an example embodiment of the IV line exiting the side gap of the more rigid outer shell and protective outer chamber from FIG. 25A to demonstrate how assembly within the shield and outer shell or removal from the shield and outer shell might occur to protect the IV joint between the female luer lock of the IV line and the male luer lock of the needleless access valve shown. As illustrated, the outer surfaces of these devices and the IV joint can be protected by a circumferential shield without the hazards of disconnecting them. It is shown that this embodiment does not require wrapping or unwrapping a tape multiple times which can lead to tangling or dislodgement of a delicate IV line with these movements.

FIG. 25D shows an example embodiment of the single IV line exiting the pod from FIG. 25C.

FIG. 25E shows an example embodiment of a needleless access valve within the shield from FIG. 25C that surrounds the outer circumference of the device. It has been assembled within the shield which provided a protective shield during assembly. The side slit can assist with placement prior to connection and assist with visualization before during and after connector assembly.

FIG. 25F shows an example embodiment of the side slit from FIG. 25E which can also be closed before, during and after assembly and can be surrounded by a more rigid clamping shell material. As illustrated is the exit of two connected IV lines from opposite ends of the pod.

FIG. 25G shows an example embodiment of how the inner chamber from FIG. 25E can be reopened for access and removal of the assembled connectors without disconnecting the IV connectors. Likewise the IV connectors and IV lines can be placed within the slit without disconnecting them.

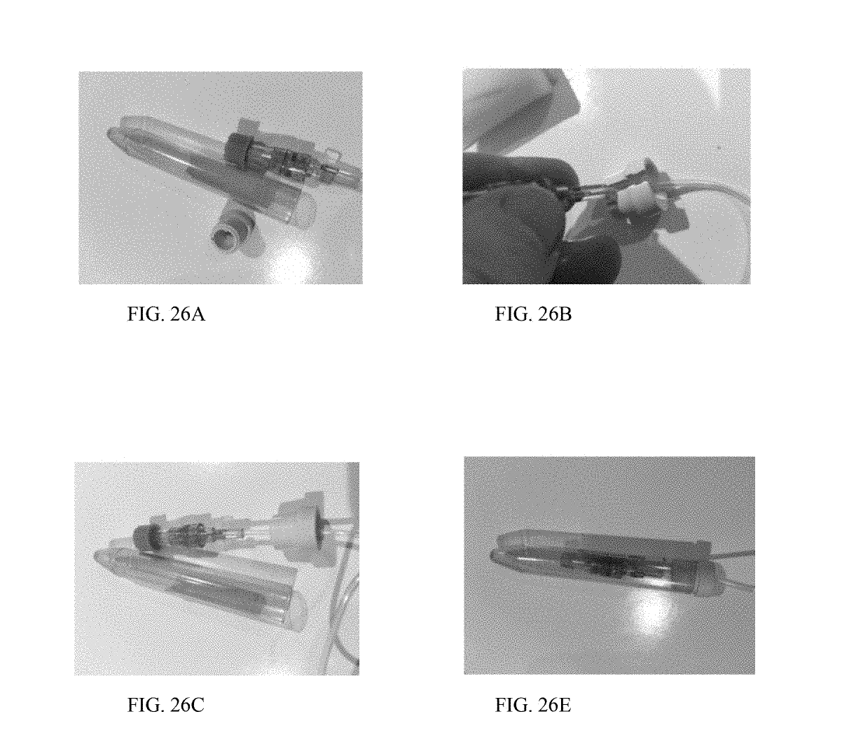

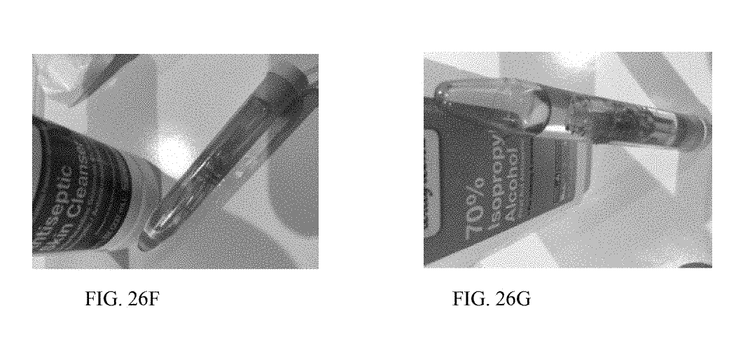

FIG. 26A shows an example embodiment of an impermeable plug surrounding an IV line attached to a needleless access valve.

FIG. 26B shows an example embodiment of how the plug from FIG. 26A can have a slit for placement of an IV line.

FIG. 26C shows an example embodiment of how the separate plug and a test tube from FIG. 26A can be positioned prior to finished assembly.

FIG. 26E shows an example embodiment of the formation of an inner chamber than encases the needleless access valve from FIG. 26A assembled to the female luer lock of the iv line and the needless access valve assembled to the cap with a male luer lock thread and a portion of the iv line. The test tube wall comprises a transparent wall so the contents within can be assessed, such as whether the inner chamber is empty, what device is enclosed, is there any visible leakage, is there any mist or solution in the inner chamber. The test tube has a relatively smaller inner diameter than the outer diameter of the plug when in a non compressed free state. Assembly creates a snug fitting and seal. The plug forms an impermeable compressible circumferential wall that contours to the outer surfaces of the enclosed IV line and the test tube forms a non absorbent impermeable rigid transparent circumferential wall.

FIG. 26F shows an example embodiment of the impermeable inner chamber from FIG. 26E retaining an antimicrobial solution such as the CHG from the labeled bottle. The devices within the inner chamber are protected by the walls of the inner chamber and the antibacterial solution during assembled storage.

FIG. 26G shows an example embodiment of the impermeable inner chamber from FIG. 26F retaining an antimicrobial solution such as the isopropyl alcohol from the labeled bottle. The devices within the inner chamber are protected by the walls of the inner chamber and the antibacterial solution which can soak outer exposed surfaces.

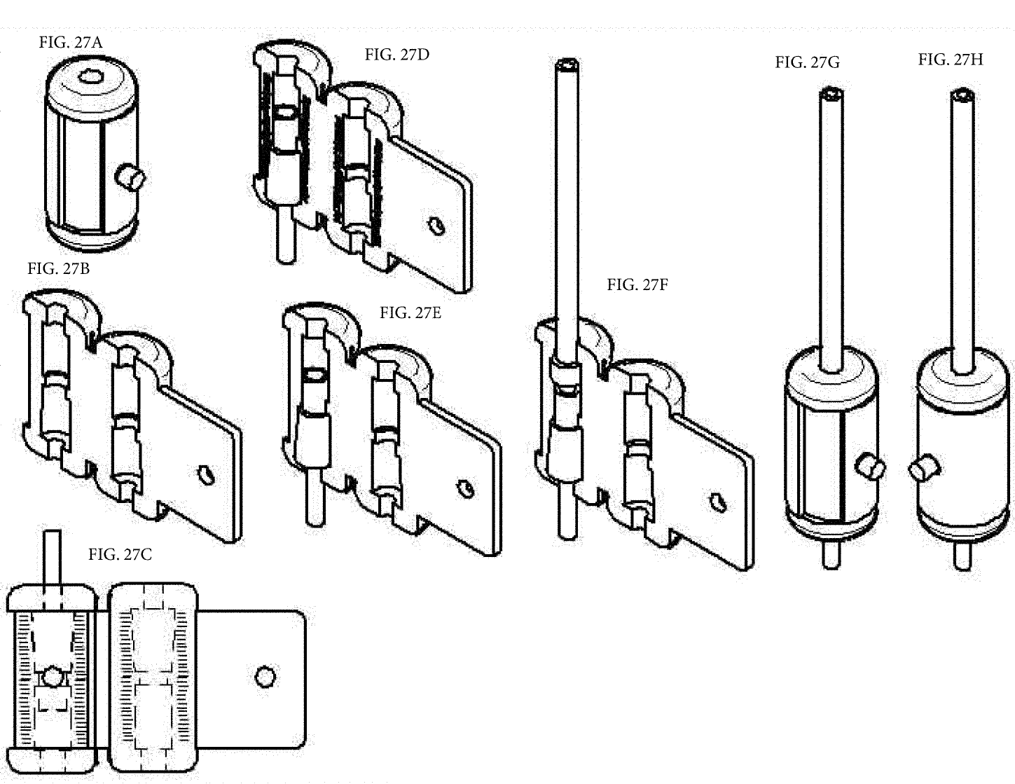

FIGS. 27A-27H show an example IV line guard, or shield as described above. As shown it can include a single component or it can have an inner foam layer surrounded by another layer. The component can fit around a connector snugly and it can have antibacterial and/or absorbent properties. It creates a sealed chamber and can self close with a built-in latch. It can include a cylindrical outer shell, an inner protective chamber, and a hinge to close around the IV line elements. FIGS. 27A, 27G and 27H show the closed state, and FIGS. 27B, 27C, 27D, 27E and 27F show the closed state. FIGS. 27F-27H show a pair of connected IV lines protected within. FIGS. 27B-27F show the inner receivers which can hold the paired IV Lines.

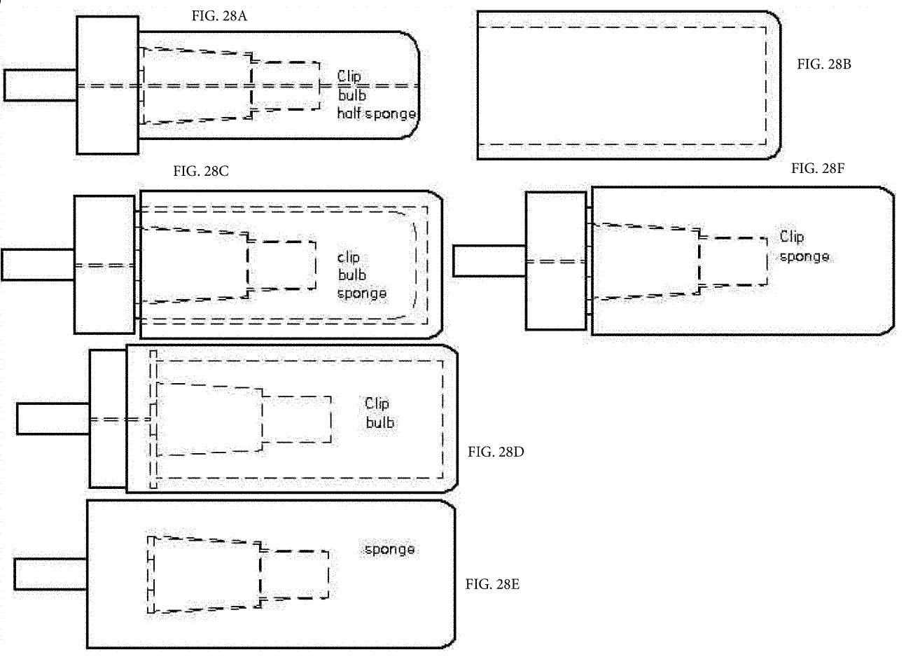

FIGS. 28A-28F show a protective outer shell with a clip for closing to halves, a sponge-like protective chamber, and a plug to contain the IV line elements entering it, such as a bulb. FIGS. 28A-28F can protect the end of an IV line element, even if not connected to a mating connector, or it can function with two paired connectors.

Although specific embodiments of the disclosure have been described, numerous other modifications and alternative embodiments are within the scope of the disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

We claim:

1. An apparatus to surround a male luer connector pre-assembled to a compatible threaded female connector, (such as a connector with a female lock connector or a needleless access valve with female luer threads), in fluid communication with an intravascular tubing entering a patient's body, to provide prolonged contamination protection to the outer surfaces of the assembled parts to prevent ingress of contamination into the inner surfaces of the intravascular tubing, the apparatus comprising:
a pod oriented around a longitudinal axis, the pod comprising:
a chamber with an inner chamber surface and an outer wall with an outer wall surface and an inner chamber length greater than the shortest combined length of a standard male luer lock and a standard female luer lock.

2. The apparatus of claim 1, wherein the chamber comprises a proximal end and a distal end and an opening thought the inner chamber surface to the outer wall surface from the proximal end toward the distal end of the chamber to allow insertion of a male luer connector pre-assembled to the compatible threaded female connector into the chamber from a position parallel to the longitudinal axis.

3. The apparatus of claim 1, further comprising t least one exit port for intravascular tubing.

4. The apparatus of claim 1, wherein the pod forms a sealed chamber around the entire length of the male luer connector and compatible female connector

5. The apparatus of claim 1, wherein the exit port seals around the IV tubing.

6. The apparatus of claim 1, wherein the chamber comprises absorbent material.

7. The apparatus of claim 1, wherein the chamber comprises permeable material.

8. The apparatus of claim 1, wherein the chamber comprises antibacterial solution or material with antibacterial properties.

9. The apparatus of claim 1, wherein the chamber is permeable to gases.

10. The apparatus of claim 1, wherein the chamber is permeable to liquids.

11. The apparatus of claim 1, wherein the chamber comprises material soaked in an antibacterial solution.

12. The apparatus of claim 1, wherein the pod comprises an end cap.

13. The apparatus of claim 1, wherein the pod comprise an end cap with an exit port.

14. The apparatus of claim 1, wherein the pod comprises an end cap without an exit port.

15. The apparatus of claim 1, wherein the pod comprises a first end cap with a port and a second end cap with a port.

16. The apparatus of claim 1, wherein the pod consists of a single monolithic part.

17. The apparatus of claim 1, wherein the pod comprises a bivalved construction.

18. The apparatus of claim 1, wherein the pod comprises a tube.

19. The apparatus of claim 1, wherein the pod comprises a pocket with a dead end.

20. The apparatus of claim 1, wherein the pod comprises a test tube.
